SEQUENCE LISTING (4 sequences)

SEQ ID NO: 1 - DNA, 1781 bp, Mus musculus
atgtacaggatgcaactcctgtcttgcattgcactaagtcttgcacttgtcacgaattcgtccttccaggtgaacccccctgagtctgaggtagctgtggccatgggcacatccctccagatcacctgcagcatgtcctgtgacgagggtgtagcccgggtgcactggcgtggtctggacaccagcttgggcagtgtacagaccctcccaggcagcagtatcctctctgtacggggcatgctgtcagacacaggcactcctgtgtgtgtgggctcctgcgggagtcgaagcttccagcactccgtgaagatccttgtgtatgccttcccagaccagctggtggtgtccccggagttccttgtacctggacaggaccaggtggtgtcctgcacggcccacaacattggcctgcagacccgaacagtctctcctttgccctgctactgggagagcagagactggagggtgcccaagccctggaaccagagcaagaagaggagatacaagaggctgagggcacaccactgttccgaatgacacaacgctggcggttaccctccctggggacccctgcccctcctgcccttcactgccaggtcaccatgcagctgcccaaactggtgctgacccatagaaaggagattccagtactacagagccagacctcacctaagccccccaacacgacctctgctgagccctacatcctgacctcatcaagtactgctgaggcagtctccactgggctcaacatcaccaccctaccttctgcccctccataccccaagcttagccctaggactctgagctctgagggaccttgccgcccgaaaatccaccaggacctggaggcaggctgggagctactctgtgaagcatcctgtgggcccggagttactgtgcgctggaccttggctcctggcgacctggcaacctaccacaagagggaggctggggcccaggcatggctaagcgtgctgcccccaggtcccatggtagagggctggttccagtgccgccaggaccctggcgggcaggtgaccaatctgtatgttcctggccaggtgaccccgaatagatctgaccccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 2 - DNA, 1734 bp, Homo sapiens
atgtacaggatgcaactcctgtcttgcattgcactaagtcttgcacttgtcacgaattcgcagtccctccaggtgaagcccctgcaggtggagcccccggagccggtggtggccgtggccttgggcgcctcgcgccagctcacctgccgcctggcctgcgcggaccgcggggcctcggtgcagtggcggggcctggacaccagcctgggcgcggtgcagtcggacacgggccgcagcgtcctcaccgtgcgcaacgcctcgctgtcggcggccgggacccgcgtgtgcgtgggctcctgcgggggccgcaccttccagcacaccgtgcagctccttgtgtacgccttcccggaccagctgaccgtctccccagcagccctggtgcctggtgacccggaggtggcctgtacggcccacaaagtcacgcccgtggaccccaacgcgctctccttctccctgctcgtcgggggccaggaactggagggggcgcaagccctgggcccggaggtgcaggaggaggaggaggagccccagggggacgaggacgtgctgttcagggtgacagagcgctggcggctgccgcccctggggacccctgtcccgcccgccctctactgccaggccacgatgaggctgcctggcttggagctcagccaccgccaggccatccccgtcctgcacagcccgacctccccggagcctcccgacaccacctccccggagcctcccaacaccacctccccggagtctcccgacaccacctccccggagtctcccgacaccacctcccaggagcctcccgacaccacctcccaggagcctcccgacaccacctcccaggagcctcccgacaccacctccccggagcctcccgacaagacctccccggagcccgccccccagcagggctccacacacacccccaggagcccaggctccaccaggactcgccgccctgagatctcccaggctgggcccacgcagggagaagtgatcccaacaggctcgtccaaacctgcgggtgaccaggccatggttagatctgacaaaactgacaaaactcacacatgcccaccgtgcccagcacctgaactcctggggggaccgtcagtcttcctcttccccccaaaacccaaggaccaactgatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctgcatgaggctctgcacaaccactacacgcagaagagcctctccctgtctccgggtaaa

SEQ ID NO: 3 - DNA, 31 bp, Artificial Sequence
  Synthetic
gacgaattcgcagtccttccaggtgaacccc

SEQ ID NO: 4 - DNA, 27 bp, Artificial Sequence
  Synthetic
gacagatctattcggggtcacctggcc

(19) United States
(12) Patent Application Publication
(10) Pub. No.: US 2011/0158982 A1
(43) Pub. Date: Jun. 30, 2011

(54) COMPOSITIONS AND METHODS FOR INHIBITING MAdCAM

(75) Inventors: Benjamin M. Segal, Ann Arbor, MI (US); Praveen Rao, Ann Arbor, MI (US)

(73) Assignee: THE REGENTS OF THE UNIVERSITY OF MICHIGAN, Ann Arbor, MI (US)

(21) Appl. No.: 12/897,532

(22) Filed: Oct. 4, 2010

Related U.S. Application Data
(60) Provisional application No. 61/248,518, filed on Oct. 5, 2009.

(51) Int. Cl. A61K 39/395; C07K 16/00; A61K 31/713; A61P 29/00; A61P 25/28

(52) U.S. Cl. 424/133.1; 530/387.3; 514/44 A

(57) ABSTRACT

The present invention relates to therapeutic targets for multiple sclerosis and neuroinflammatory diseases and injuries. In particular, the present invention relates to targeting MAdCAM in the treatment of such disorders.

### CROSS REFERENCE TO RELATED APPLICATIONS

This application claims priority to application Ser. No. 61/248,518, filed Oct. 5, 2009, which is herein incorporated by reference in its entirety.

### FIELD OF THE INVENTION

The present invention relates to therapeutic targets for multiple sclerosis and neuroinflammatory diseases and injuries. In particular, the present invention relates to targeting MAdCAM in the treatment of such disorders.

### BACKGROUND OF THE INVENTION

Multiple sclerosis (MS) is the most common non-traumatic cause of neurological disability among young adults in Western nations. It currently afflicts 400,000 individuals in the United States and more than 1,000,000 individuals worldwide. Over the past two decades the United States Food and Drug Administration (FDA) has approved six disease modifying therapies for the long term treatment of individuals with RRMS. These therapies include three different formulations of recombinant interferonβ and glatiramer acetate. Double-blind, placebo-controlled, randomized trials have demonstrated that each of the formulations of interferonβ as well as glatiramer acetate reduce the frequency of clinical exacerbations by approximately 30% and significantly suppress the incidence of gadolinium enhancing lesions detected by serial magnetic resonance imaging. Furthermore, retrospective epidemiological studies indicate that maintenance therapy with interferonβ slows the rate of accumulation of disability in RRMS. Despite these accomplishments in MS therapeutics, the currently available therapies are only modestly effective. None are curative and most patients continue to transition into secondary progression despite treatment. More aggressive approaches with globally immunosuppressive chemotherapies are beleaguered by serious adverse side effects, including an increased risk of neoplasia and infection and, in the case of mitoxantrone, cardiotoxicity.

Thus, development of new therapeutic targets and agents is needed.

### SUMMARY OF THE INVENTION

The present invention relates to therapeutic targets for multiple sclerosis and neuroinflammatory diseases and injuries. In particular, the present invention relates to targeting MAdCAM in the treatment of such disorders.

For example, in some embodiments, the present invention provides a pharmaceutical composition comprising an FC-Fusion protein that inhibits at least one activity (e.g., blocks the binding to its ligand) of a mucosal addressin cell adhesion molecule-1 (MAdCAM) protein. In some embodiments, the FC-fusion protein has the amino acid sequence of SEQ ID NOs: 1 or 2. In some embodiments, the composition reduces or eliminates symptoms or prevents relapses of a neuroinflammatory condition (e.g., MS).

In further embodiments, the present invention provides a method of treating a neuroinflammatory condition, comprising: administering a composition that inhibits at least one activity of a MAdCAM protein to a subject diagnosed with a neuroinflammatory condition (e.g., MS) under conditions such that symptoms of the neuroinflammatory condition are reduced or eliminated. In some embodiments, the composition is a MAdCAM FC-Fusion, an anti-MAdCAM antibody, a small molecule, an siRNA that inhibits the expression of MAdCAM or its ligand (α4β7 integrin) or an antisense nucleic acid that inhibits the expression of MAdCAM or its ligand (α4β7 integrin). In some embodiments, the composition is a MAdCAM FC-Fusion (e.g., having the amino acid sequence of SEQ ID NOs: 1 or 2). In some embodiments, the composition accelerates recovery of the subject from symptoms of the neuroinflammatory condition or prevents relapses of symptoms of the neuroinflammatory condition.

### DESCRIPTION OF THE FIGURES

FIG. 1 shows the molecular structure of MAdCAM-1. MAdCAM-1 is composed of two immunoglobulin domains (Ig1 and Ig2) at the amino terminus, separated by a third immunoglobulin domain (Ig3) by a mucin-like domain.

FIG. 2 shows that α4β7 integrin, the counter-receptor of MadCAM-1, is preferentially expressed on myelin-specific CD4⁺ Th17 cells. Naive CD4+ T cells expressing a transgenic T cell receptor specific for MOG₃₅₋₅₅ (A) or MBP_{Ac1-11} (B) were cultured with APCs and myelin peptide under Th1, Th2 or Th17 polarizing conditions. Three days later the cells were harvested, washed and stained with fluorochrome-conjugated monoclonal antibodies specific for activated α4β7 integrin and CD4. CD4⁺ gated cells were analyzed for level of α4β7 integrin expression by flow cytometry. Background staining of Th17 polarized cells that were incubated with an irrelevant isotype matched control antibody is illustrated.

FIG. 3 shows that Th17 and Th1, but not Th2, 2D2 cells accumulate in the CNS during EAE. (A) Naïve (CD62L^{hi}, CD44^{lo}, CD25^{-/-}) CD4³⁰ 2D2 cells were cultured with MOG₃₅₋₅₅ under Th1, Th2 or Th17 polarizing conditions. (B) Absolute numbers of donor cells/organ were calculated by multiplying cell yield by percent of CD45.2⁺CD4⁺ donor cells.

FIG. 4 shows that MAdCAM-1 is upregulated on inflamed blood vessels in the CNS of mice with EAE. Sections of spinal cords harvested from PLP₁₃₉₋₁₅₁ immunized mice during acute EAE were stained with FITC-conjugated anti-CD45 and PE-conjugated anti-MAdCAM-1 monoclonal antibodies. A representative image of an active lesion is shown.

FIG. 5 shows that administration of anti-MAdCAM-1 mAbs accelerate recovery from the first episode of EAE. Mean clinical scores between the groups were significantly different on days 15-20 (*p<0.05; Student's T test assuming unequal variances). The arrows denote the days on which the antibodies were given.

FIG. 6 shows that anti-MAdCAM-1 monoclonal antibodies, administered during remissions of EAE, prevent subsequent relapse. Mean clinical scores between the groups were significantly different on days 27-35 (*p<0.05). The arrows denote the days on which the antibodies were given.

FIG. 7 shows the construction and characterization of MAdCAM-1-Fc fusion protein. (A) Map of the plasmid p-FUSE-mIgG2a-Fc2a. (B) Western blot analysis of supernatants (rows 1-3) and extracts (4-6) of untransfected HEK293T cells (1, 4) or of HEK293 T cells transfected with empty vector (2, 5) or with the plasmid containing the extracellular domain of MAdCAM-1-Fc (3, 6).

FIG. 8 shows that treatment of mice with MAdCAM-Fc fusion protein suppresses the subsequent development of EAE. Mice were treated with either MAdCAM-Fc fusion protein (open triangles) or control (unfused) Fc protein (closed circles) between days 6 and 15 post-immunization (0.5 mg/mouse every 3 days). Mean clinical scores were significantly different on the days indicated (*p<0.05).

FIG. 9 shows that treatment of mice with MAdCAM-Fc fusion protein during the first remission of EAE prevents subsequent relapse. At the time of remission (day 23), the mice were divided into two clinically comparable groups. One group was treated with MAdCAM-Fc fusion protein (open circles) and the other with control (unfused) Fc protein (0.5 mg/mouse every 3 days between days 21 and 33. Mean clinical scores were significantly different on the days indicated (*p<0.05).

FIG. 10 shows that treatment with anti-MAdCAM-1 suppresses EAE induced by the adoptive transfer of encephalitogenic Th17 cells. (A) Mice were rated for degree of paralysis by an observer blinded to the treatment of each group. Mean clinical scores are shown from the day of transfer until sacrifice. * control group is significantly more debilitated than the anti-MAdCAM treated group, p<0.05. (B) Representative spinal cord sections of a mouse in the anti-MAdCAM-1 treated group (left) and the control group (right). Sections were stained with hematoxylin and eosin. Magnification ×4.

FIG. 11 shows that human MadCAM-FC accelerates recovery from acute EAE when administered follow disease onset.

FIG. 12 shows that Human MAdCAM-Fc suppresses EAE induced by active immunization.

FIG. 13 shows the sequence of mouse (A) and human (B) pFUSE-mlgG2A-Fc2-Madcam1. Underlined sequences: Begins from the ATG of the IL-2 signal sequence and contains the remainder of the multiple cloning sequence site. The MADCAM portion has been cloned in between the signal sequence and the Fc portion for both sequences. The shaded sequences: Sequences for the mouse and human MADCAM-1 respectively. The signal sequences and the transmembrane portion of both the genes were left out while cloning. The double underlined have the Fc portion of the mouse and human Fc plasmids respectively from the first amino acid till the last amino acid of the Fc portion.

FIG. 14 shows the effect of wild type and mutant MADCAM-FC on EAE.

### DEFINITIONS

To facilitate an understanding of the present invention, a number of terms and phrases are defined below:

As used herein, the term “neuroinflammatory condition” refers to any condition (e.g., injury) or disease that results in inflammation of a neurological tissue. Examples include, but are not limited to, multiple sclerosis (MS), acute trauma (e.g., head or spinal cord injury), Alzheimer's disease, amyotrophic lateral sclerosis (ALS, also known as Lou Gehrig's disease), acute disseminated encephalomyelitis, Bell's palsy, neurosarcoidosis, CNS complications of collagen vascular diseases (including systemic lupus erythematosus, Sjogren's disease, takayasu arteritis, temporal/giant cell arteritis, granulomatosis and poyarteritis nodosa), primary CNS angiitis, transverse myelitis, Susac's syndrome, PANDAS (pediatric autoimmune neuropsychiatric disorders associated with streptococcal infections), sydenham's chorea, adrenomyeloneuropathy, Guillain-Barre syndrome, chronic inflammatory demyelinating polyneuropathy, polymyositis, neurobehcet's disease, paraneoplastic syndromes, limbic encephalitis, Lambert-Eaton myasthenic syndrome and myasthenia gravis.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “non-human animals” refers to all non-human animals including, but are not limited to, vertebrates such as rodents, non-human primates, ovines, bovines, ruminants, lagomorphs, porcines, caprines, equines, canines, felines, ayes, etc.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment is retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under ‘medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

As used herein, the term “siRNAs” refers to small interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to, or substantially complementary to, a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “epitope” as used herein refers to that portion of an antigen that makes contact with a particular antibody.

When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

“Amino acid sequence” and terms such as “polypeptide” or “protein” are not meant to limit the amino acid sequence to the complete, native amino acid sequence associated with the recited protein molecule.

The term “native protein” as used herein to indicate that a protein does not contain amino acid residues encoded by vector sequences; that is, the native protein contains only those amino acids found in the protein as it occurs in nature. A native protein may be produced by recombinant means or may be isolated from a naturally occurring source.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., neuroinflammatory disease). Test compounds comprise both known and potential therapeutic compounds. A test compound can be determined to be therapeutic by screening using the screening methods of the present invention. In some embodiments of the present invention, test compounds include antisense compounds.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₄⁺, wherein W is Cᵢ₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

### DETAILED DESCRIPTION OF THE INVENTION

The present invention relates to therapeutic targets for multiple sclerosis and neuroinflammatory diseases and injuries. In particular, the present invention relates to targeting MAdCAM in the treatment of such disorders.

The approval of natalizumab (Tysabri) by the FDA in 2004 signified a critical advancement in the immunomodulation of RRMS. Natalizumab is a humanized IgG4κ monoclonal antibody that is specific for alpha-4 (α4) integrin. It is believed to act by blocking the adhesion of leukocytes to the endothelial lining of CNS vessels, thereby preventing their passage across the blood-brain-barrier to reach the CNS. In a Phase 3 trial, natalizumab monotherapy, administered as monthly intravenous infusions, reduced MS relapses by 68% and new lesion formation by 78% compared to placebo, a margin far greater than that seen in previous trials of interferon β and glatiramer acetate. Other therapeutic benefits of natalizumab included an increase in the percentage of disease-free individuals, a reduction in hospitalizations and steroid use for MS exacerbations, significant improvements in assessments of health-related quality of life, and relative preservation of cognitive functions and vision compared to the placebo group. However, natalizumab was soon withdrawn from the market after it was linked to two cases of progressive multifocal leukoencephalopathy (PML) when administered in combination with interferon β. After a review of safety information, the drug was reintroduced to the US market in 2006 under a special restricted distribution program, the TOUCH Prescribing Program.

PML is a rare opportunistic infection caused by the JC virus that typically occurs in patients who are immunocompromised as the result of AIDS or multimodal chemotherapeutic treatment given to prevent organ transplant rejection. In those settings it is almost universally fatal. PML results in CNS demyelination and, hence, can be difficult to distinguish from MS progression based on its clinical presentation and early radiological appearance. Therefore, health professionals must exercise a high level of vigilance when monitoring MS patients who are treated with natalizumab in order to diagnose PML early and withdraw the drug in an attempt to avert severe disability or death. As of the present, PML has occurred in 3 patients who received natalizumab in clinical trials and in an additional 6 out of 6,800 patients treated with the drug for 2 years or longer since its reintroduction to the market in July 2006.

There are no known interventions that can reliably prevent or cure PML. It is not known whether early detection of PML and a course of plasma exchange to remove natalizumab from the blood will mitigate the disease. Every MS patient who has thus far developed PML during treatment with natalizumab did so after taking the drug for at least 12 months, with the average length of treatment being 19 months. The reason for this latency and the precise relationship between the risk of PML and the duration of treatment are unknown. As there is limited experience with long term exposure, concerns have been voiced that the incidence of PML and other opportunistic infections might continue to rise beyond 2 years of therapy. In addition to the risk of PML, two MS patients have developed melanoma during treatment with natalizumab, although the ultimate link between the drug and cancer remains unclear. In the Phase 3 trail of natalizumab monotherapy, patients on treatment had an increased incidence of urinary tract infection and pneumonia. In the post-marketing experience, one patient who received natalizumab developed herpes encephalitis and died; another patient developed herpes meningitis and recovered.

In PML, JC virus that persists in a latent form in B cells and the kidney after primary infestion, becomes reactivated and causes demyelination in the brain. A number of viral pathogens can establish latency within the human CNS, including members of the herpesvirus (HSV, VZV), retrovirus (HIV), and polyomavirus (JC virus) families. Reactivation of these viruses after latency causes a spectrum of neurological disorders such as herpes simplex encephalitis, shingles, HIV-associated dementia, and PML. The present invention is not limited to a particular mechanism. Indeed, an understanding of the mechanism is not necessary to practice the present invention. Nonetheless, it is contemplated that natalizumab is associated with reactivation of herpes viruses and/or retroviruses.

CNS viral latency models have been investigated in mice, including those caused by alphaviruses and flaviviruses. In these models, initial control of virus replication occurs once the host mounts an antigen-specific immune response against viral determinants allowing for clinical recovery. However, viral nucleic acids persist in target cells over the long term. Prevention of virus reactivation requires ongoing immune surveillance of CNS tissues, and recurrent disease can occur in an immunocompromised state.

The present invention is not limited to a particular mechanism. Indeed, an understanding of the mechanism is not necessary to practice the present invention. Nonetheless, it is contemplated that the putative mechanism of action of natalizumab in ameliorating MS is to bind and inhibit the functional activity of α4β1 integrin (VLA-4) on the surface of encephaliotogenic CD4⁺ T cells and other leukocytes that participate in the formation of the perivascular CNS infiltrates associated with demyelination and axonal damage. Interactions between VLA-4 and its endothelial ligand, VCAM-1, mediate firm adherence of leukocytes to the luminal surface of vessel walls, a requisite step for their extravasation into inflamed tissues. Engagement of VLA-4 also triggers leukocyte activation and expression of chemokine receptors that further facilitate diapedesis. Experiments in the animal model, experimental autoimmune encephalomyelitis (EAE), provided the initial proof of principle that α4 dependent interactions are important for the infiltration of leukocytes into CNS parenchyma and the initiation of demyelinating lesions. Hence, administration of monoclonal antibodies specific for α4 prevented clinical EAE. However, in addition to associating with the β1 chain to form VLA-4, α4 integrin complexes with the β7 integrin chain resulting in a distinct adhesion molecule, α4β7. The present invention is not limited to a particular mechanism. Indeed, an understanding of the mechanism is not necessary to practice the present invention. Nonetheless, it is contemplated that the therapeutic efficacy of Tysabri, and α4 binding reagents in general, is secondary to blockade of one or both of the α4 heterodimers.

The major ligand for α4β7 is mucosal addressin cell adhesion molecule-1 (MAdCAM-1). MAdCAM-1 is a 60-kDa multidomain type-1 membrane glycoprotein, comprised of immunoglublin-like (Ig) and muclin-related sequences and a short cytoplasmic region. Both mouse and human MAdCAM-1 mRNA expression is restricted to small intestine, colon, spleen, pancreas and brain, as assessed by Northern blotting. On the protein level, it is constitutively expressed on high endothelial venules in the mesenteric lymph nodes and Peyer's patches and on postcapillary venules in the lamina propia.

Naive lymphocytes express modest levels of α4β7, whereas effector/memory cells can be subdivided into α4β7^{high} and α4β7^{low} subsets, with the α4β7^{high} populations preferentially binding to MAdCAM-1. Under physiological conditions, α4β7⁺ lymphocytes preferentially migrate to the gut, whereas α4β1⁺ lymphocytes migrate to extraintestinal tissues. Studies using knock-out mice and blocking antibodies have demonstrated a critical role for MAdCAM-1/α4β7 interactions in the homing of both naive and memory/effector lymphocytes to the gut and gut associated lymphoid tissues during homeostasis and in the context of intestinal inflammation. In Peyer's patches, initial capture from blood flow is mediated by L-selectin, and subsequent α4β7-mediated adhesion is required to convert rolling to arrest. In contrast, homing of effector and memory lymphocytes to the lamina propria does not require L-selectin and is solely dependent on interactions between MAdCAM-1 and α4β7.

The ectodomain of MAdCAM-1 contains two N-terminal Ig-domains followed by a mucin-like sequence, and a third (membrane proximal) Ig domain. The N-terminal Ig domains show high homology to the related vascular integrin ligands, VCAM-1 and ICAM-1. Collectively they contain the high affinity α4β7 binding site. The second and third Ig domains of MAdCAM-1 are separated by a 37 amino acid residue serine/thronine-rich, mucin-like domain that can be modified by addition of L-selectin-binding glycoconjugates and has been implicated in L-selectin dependent lymphocyte rolling. The third Ig domain of MAdCAM shares homology with the constant region of IgA1. The unique combination of integrin-binding and mucin-like structural elements allows MAdCAM-1 to participate and regulate both L-selectin and α4β7 integrin-mediated interactions in lymphocytes. The structurally related vascular adhesion receptors ICAM-1 and VCAM-1 preferentially bind to the lymphocyte counter-receptors LFA1/αLβ2 (ICAM-1) and VLA4/α4β1 or α4β7 (VCAM-1).

Migration of circulating leukocytes from the blood to sites of inflammation is a multistep process that involves initial tethering to and rolling along the vascular endothelial surface, leukocyte stimulation, firm adhesion, and finally migration across the endothelium. The α4-integrins, α4β1 and α4β7 are most prominent on mononuclear leukocytes and have a unique role in this multistep cascade. Alpha-4 integrins and their endothelial counter receptors, VCAM-1 and MAdCAM-1, mediate both rolling (when the integrins are in a low-affinity state) and arrest (when the integrins are in a high-affinity state). α4β1 mediates cell adhesion to VCAM-1 and to an alternatively spliced form of the extracellular matrix protein fibronectin (FN). α4β7 mediates binding to MAdCAM-1, VCAM-1, and FN.

The molecular basis of the interactions of α4β7 and α4β1 with the endothelial cell adhesion molecules MAdCAM-1 and VCAM-1 has been studied in detail. Because ICAM-1 is structurally related to MAdCAM but does not bind to α4β7 integrin, chimeric MAdCAM-1/ICAM constructs were used to map the binding site of MAdCAM-1 to α4β7. These studies revealed that the two N-terminal domains of MAdCAM-1 are sufficient to confer α4β7 binding comparable to the full-length ectodomain of wild-type MAdCAM-1. The first Ig domain of MAdCAM-1 (Ig1) supports binding of activated α4β7 integrin (in the presence of Mn²⁺), but not inactive α4β7 (absence of Mn²⁺). Replacement of the first Ig domain of MAdCAM by the corresponding domain of ICAM-1 abolishes binding of α4β7. Together, these domain swapping experiments indicate that the first Ig domain of MAdCAM-1 is necessary and sufficient for α4β7 binding and that sequences within the second Ig domain of MAdCAM-1 reinforce this interaction, either by providing additional contact points for the integrin association or, indirectly, through conformational changes. The ability of the N-terminal two Ig domains of MAdCAM-1 to mediate α4β7 binding similar to full-length ectodomain indicates that that the mucin and neighboring IgA1 homologous sequences are not necessary for this interaction. Splice variants of MAdCAM-1 have been found in both murine and human tissues that selectively lack the mucin and IgA1 homologous sequences. The truncated (two Ig domain) form of MAdCAM-1 is predicted to retain the α4β7 binding ability.

Site-directed mutagenesis and molecular modeling was used to map and define amino acid residues on MAdCAM-1 that participate in the association with α4β7 integrin. A linear binding motif consisting of three residues (L40, D41, and T42) within the C-D loop of the first Ig domain of MAdCAM-1 is of importance for binding. Mutation of any of these three residues completely abolishes binding of lymphoid cell lines expressing either wild-type or constitutively active α4β7 to the MAdCAM ectodomain (MAdCAM-1-Fc) immobilized to ELISA plates. Binding of the lymphoid cells to MAdCAM-1-Fc was completely inhibited by Abs specific for the integrin α4 chain, indicating that no other molecules on the cell surface contributed to adhesion. In the presence of suboptimal levels of anti-CD3 Ab, the immobilized wild-type MAdCAM-1 ectodomain enhances mononuclear lymphocyte proliferation in a dose-dependent manner. In the same assay, immobilized MAdCAM-1 molecules with mutations in the α4β7 binding site (L40, D41, or T42) do not exhibit any co-stimulatory activity, indicating that MAdCAM-1 enhanced lymphocyte activation critically depends on its interaction with α4β7. Residues L40, D41, and T42 are conserved between mouse and human MAdCAM-1, demonstrating an evolutionary conserved binding mechanism.

In two previous publications administration of monoclonal antibodies directed against MAdCAM-1 or the β7 chain prevented EAE induced by active immunization and adoptive transfer, respectively. In addition, myelin-specific T cells derived from β7 deficient mice were impaired in their ability to infiltrate the CNS and induce disease. Nevertheless, other researchers recently reported a selective inability of β1 deficient T cells to accumulate in the CNS when EAE was induced in bone marrow chimeric mice containing a combination of β1-positive and β1-negative T cells in the periphery. Earlier studies also attest to the therapeutic efficacy of anti-VCAM-1 antibodies in EAE. Collectively these data show that both α4β7 and α4β1 play important roles in the pathogenesis of autoimmune demyelination and that they are not completely redundant in their functions.

The advantage of using agents that specifically block α4β7/MAdCAM-1, as opposed to VLA-4NCAM-1, in the clinical setting is that the immunosuppressive effects of the former are more targeted and, therefore, less likely to interfere with protective immune responses. Hence, whereas VLA-4 is universally expressed on T cells, B cells, monocytes and eosinophils, expression of α4β7 is restricted to a subset of activated T cells, B cells and eosinophils. Similarly, VCAM-1 is widely expressed on endothelial cells throughout the body, while MAdCAM-1 expression is normally restricted to the vasculature supplying the gut. Embodiments of the present invention provide reagents that specifically block the activity of α4β7, as opposed to all α4 heterodimers. It is contemplated that such agent prevents exacerbations of inflammatory demyelination with less of an impact on antimicrobial immunity and, in particular, immunosurveillance against latent viruses. Thus, embodiments of the present invention provide novel, highly effective and targeted immunotherapies for the treatment of autoimmune demyelinating diseases such as MS that pose less of a risk for the development of opportunistic infections or neoplasms than currently available drugs.

### I. MAdCAM Targeted Therapeutics

In some embodiments, the present invention provides compositions and methods for targeting MAdCAM-α4β7 interactions. The present invention is not limited to a particular MAdCAM targeted therapy. Exemplary therapeutic compositions and methods are described below.

A. FC-Fusion Therapy

In some embodiments, MAdCAM targeting therapeutics are Fc-Fusion proteins. Fc-fusion proteins are chimeric proteins comprising the effector region of a protein (e.g., MAdCAM), fused to the Fc region of an immunoglobulin G (IgG).

The Fc region mediates effector functions, such as antibody-dependent cellular cytotoxicity (ADCC) and complement-dependent cytotoxicity (CDC). In ADCC, the Fc region of an antibody binds to Fc receptors (FcγRs) on the surface of immune effector cells such as natural killers and macrophages, leading to the phagocytosis or lysis of the targeted cells.

In CDC, the antibodies kill the targeted cells by triggering the complement cascade at the cell surface. IgG isoforms exert different levels of effector functions increasing in the order of IgG4<IgG2<IgG1≦IgG3. Human IgG1 displays high ADCC and CDC.

The MAdCAM fragment of the Fc fusion molecule can be any suitable MAdCAM fragment. In some embodiments, the fragment is an extracellular domain of MAdCAM. Alternatively, a variant of a MAdCAM fragment described above can be used. Desirably, the variant of the MAdCAM fragment retains the functionality of the selected fragment. A variant of a MAdCAM fragment can be obtained by any suitable method, including random and site-directed mutagenesis of the nucleic acid encoding the MAdCAM fragment (see, e.g., Walder et al., Gene, 42, 133 (1986); Bauer et al., Gene, 37, 73 (1985); U.S. Pat. Nos. 4,518,584 and 4,732,462; and QuikChange Site-Directed Mutagenesis Kit (Stratagene, LaJolla, Calif.); each of which is herein incorporated by reference in its entirety). While a variant of the nucleic acid can be generated in vivo and then isolated and purified, alternatively, a variant of the nucleic acid can be synthesized. Various techniques used to synthesize nucleic acids are known in the art (see, e.g., Lemaitre et al., Proc. Natl. Acad. Sci., 84, 648-652 (1987)).

Additionally, a variant can be synthesized using peptide-synthesizing techniques known in the art (see, e.g., Bodansky, Principles of Peptide Synthesis, Springer-Verlag, Heidelberg, 1984). In particular, a (poly)peptide can be synthesized using the procedure of solid-phase synthesis (see, e.g., Merrifield, J. Am. Chem. Soc., 85, 2149-54 (1963); Barany et al., Int. J. Peptide Protein Res., 30, 705-739 (1987), and U.S. Pat. No. 5,424,398; each of which is herein incorporated by reference in its entirety). If desired, a (poly)peptide can be synthesized with an automated peptide synthesizer. Removal of the t-butyloxycarbonyl (t-BOC) or 9-fluorenylmethyloxycarbonyl (Fmoc) amino acid blocking groups and separation of the (poly)peptide from the resin can be accomplished by, for example, acid treatment at reduced temperature. The (poly)peptide-containing mixture can then be extracted, for instance, with dimethyl ether, to remove non-peptidic organic compounds, and the synthesized (poly)peptide can be extracted from the resin powder (e.g., with about 25% w/v acetic acid). Following the synthesis of the (poly)peptide, further purification (e.g., using high performance liquid chromatography (HPLC)) optionally can be done in order to eliminate any incomplete (poly)peptides or free amino acids. Amino acid and/or HPLC analysis can be performed on the synthesized polypeptide to determine its identity. The (poly)peptide can be produced as part of a larger fusion protein, such as by the above-described methods or genetic means, or as part of a larger conjugate, such as through physical or chemical conjugation.

The variant of the above-described MAdCAM fragment includes molecules that have about 50% or more identity to the above-described MAdCAM fragments. Preferably, the variant includes molecules that have 75% identity to the above-described MAdCAM fragments. More preferably, the variant includes molecules that have 85% (e.g., about 90% or more, about 95% or more, about 96% or more, about 97% or more, about 98% or more, or about 99% or more) identity with the above-described MAdCAM fragments. Ideally, the variant of the MAdCAM fragment contains from 1 to about 40 (e.g., about 5, about 10, about 15, about 20, about 25, about 30, about 35, or ranges thereof) amino acid substitutions, deletions, inversions, and/or insertions thereof. More preferably, the variant of the above-described MAdCAM fragments contains from 1 to about 20 amino acid substitutions, deletions, inversions, and/or insertions thereof. Most preferably, the variant of the scFv fragment contains from 1 to about 10 amino acid substitutions, deletions, inversions, and/or insertions thereof.

The substitutions, deletions, inversion, and/or insertions of the MAdCAM fragment preferably occur in non-essential regions. The identification of essential and non-essential amino acids in the MAdCAM fragment can be achieved by methods known in the art, such as by site-directed mutagenesis and AlaScan analysis (see, e.g., Moffison et al., Chem. Biol. 5(3), 302-307 (2001)). Essential amino acids should be maintained or replaced by conservative substitutions in the variants of the MAdCAM fragments. Non-essential amino acids can be deleted, or replaced by a spacer or by conservative or non-conservative substitutions.

The variants can be obtained by substitution of any of the amino acids as present in the MAdCAM fragment. As can be appreciated, there are positions in the sequence that are more tolerant to substitutions than others, and some substitutions can improve the activity of the native MAdCAM fragment. The amino acids that are essential should either be identical to the amino acids present in the MAdCAM fragment, or substituted by conservative substitutions. The amino acids that are nonessential can be identical to those in the MAdCAM fragment, can be substituted by conservative or non-conservative substitutions, and/or can be deleted.

Conservative substitution refers to the replacement of an amino acid in the MAdCAM fragment with a naturally or non-naturally occurring amino acid having similar steric properties. Where the side-chain of the amino acid to be replaced is either polar or hydrophobic, the conservative substitution should be with a naturally or non-naturally occurring amino acid that is also polar or hydrophobic (in addition to having the same steric properties as the side-chain of the replaced amino acid). When the native amino acid to be replaced is charged, the conservative substitution can be with a naturally or non-naturally occurring amino acid that is charged, or with a non-charged (polar, hydrophobic) amino acid that has the same steric properties as the side-chains of the replaced amino acid. For example, the replacement of arginine by glutamine, aspartate by asparagine, or glutamate by glutamine is considered to be a conservative substitution.

In order to further exemplify what is meant by conservative substitution, Groups A-F are listed below. The replacement of one member of the following groups by another member of the same group is considered to be a conservative substitution.

Group A includes leucine, isoleucine, valine, methionine, phenylalanine, serine, cysteine, threonine, and modified amino acids having the following side chains: ethyl, iso-butyl, —CH2CH2OH, —CH2CH2CH2OH, —CH2CHOHCH3 and CH2SCH3.

Group B includes glycine, alanine, valine, serine, cysteine, threonine, and a modified amino acid having an ethyl side chain.

Group C includes phenylalanine, phenylglycine, tyrosine, tryptophan, cyclohexylmethyl, and modified amino residues having substituted benzyl or phenyl side chains.

Group D includes glutamic acid, aspartic acid, a substituted or unsubstituted aliphatic, aromatic or benzylic ester of glutamic or aspartic acid (e.g., methyl, ethyl, n-propyl, iso-propyl, cyclohexyl, benzyl, or substituted benzyl), glutamine, asparagine, CO—NH-alkylated glutamine or asparagine (e.g., methyl, ethyl, n-propyl, and iso-propyl), and modified amino acids having the side chain —(CH2)3COOH, an ester thereof (substituted or unsubstituted aliphatic, aromatic, or benzylic ester), an amide thereof, and a substituted or unsubstituted N-alkylated amide thereof.

Group E includes histidine, lysine, arginine, N-nitroarginine, p-cycloarginine, g-hydroxyarginine, N-amidinocitruiine, 2-amino guanidinobutanoic acid, homologs of lysine, homologs of arginine, and ornithine.

Group F includes serine, threonine, cysteine, and modified amino acids having C1-C5 straight or branched alkyl side chains substituted with —OH or —SH.

A non-conservative substitution is a substitution in which the substituting amino acid (naturally or non-naturally occurring) has significantly different size, configuration and/or electronic properties compared with the amino acid being substituted. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cyclohexylmethyl glycine for alanine, or isoleucine for glycine. Alternatively, a functional group can be added to the side chain, deleted from the side chain or exchanged with another functional group. Examples of nonconservative substitutions of this type include adding an amine, hydroxyl, or carboxylic acid to the aliphatic side chain of valine, leucine or isoleucine, or exchanging the carboxylic acid in the side chain of aspartic acid or glutamic acid with an amine or deleting the amine group in the side chain of lysine or ornithine.

For non-conservative substitutions, the side chain of the substituting amino acid can have significantly different steric and electronic properties from the functional group of the amino acid being substituted. Examples of such modifications include tryptophan for glycine, and lysine for aspartic acid.

The Fc region of the fusion molecule can be any suitable Fc region of an antibody. Preferably, the Fc region increases the stability, decreases the clearance time of the peptide or polypeptide (e.g., MAdCAM fragment) from plasma and tissues, thereby enabling a minimum effective dose to be realized. The Fc region of an antibody is limited in variability and is responsible for the biological effector function of the antibody, which is designed to bring about the destruction of the target recognized as foreign by the peptide or polypeptide (e.g., MAdCAM fragment). The Fc portion varies between antibody classes (and subclasses) but is identical within that class. If the Fc region is a human Fc region, the Fc region is selected from the classes of IgA, IgD, IgE, IgG, and IgM. If the Fc region is an IgA or IgG Fc region, the subclass is selected from IgA1 and IgA2, or IgG1, IgG2, IgG3, and IgG4, respectively. In some embodiments, the Fc region is an Fc region of IgG. In some embodiments, the Fc region is an IgG2A heavy chain (CH2 and CH3 domains and the hinge region).

The peptide or polypeptide (e.g., MAdCAM fragment) and Fc region of the fusion molecule optionally are joined together by a linker. The linker can be any suitable long flexible linker. The linker can be any suitable length, but is preferably at least about 15 (e.g., at least about 20, at least about 25, at least about 30, at least about 35, at least about 40, at least about 45, at least about 50, or ranges thereof) amino acids in length. Preferably, the long flexible linker is an amino acid sequence that is naturally present in immunoglobulin molecules of the host, such that the presence of the linker would not result in an immune response against the linker sequence by the mammal.

The generation of the fusion molecules of the invention is within the ordinary skill in the art, and can comprise the use of restriction enzyme or recombinational cloning techniques (see, e.g., Gateway™ (Invitrogen), Invivogen and U.S. Pat. No. 5,314,995; herein incorporated by reference in its entirety).

As discussed above, the Fc fusion molecules encompassed by the invention comprise a MAdCAM fragment, an Fc region, and, optionally, a flexible linker. Further, any of these fusion molecules can be expressed with a suitable leader sequence, which leader sequence specifies how the fusion is trafficked through a cell expressing the leader-fusion polypeptide. In some embodiments, fusion protein include a signal sequence such as an IL-2 signal sequence (IL-2ss).

Variants of the Fc fusion molecules can be obtained by any suitable method, including those methods discussed above. The variants of the above-described Fc fusion molecules include molecules that have about 90% or more percent identity (e.g., about 95% or more, about 96% or more, about 97% or more, about 98% or more, or about 99% or more) with the above-described Fc fusion molecules. Preferably, the variants of the Fc fusion molecules contain from 1 to about 50 (e.g., about 5, about 10, about 15, about 20, about 25, about 30, about 35, about 40, about 45, or ranges thereof) amino acid substitutions, deletions, inversions, and/or insertions thereof. More preferably, the variants contain from 1 to about 30 amino acid substitutions, deletions, inversions, and/or insertions thereof. Most preferably, the variants contain from 1 to about 20 amino acid substitutions, deletions, inversions, and/or insertions thereof. Ideally, the variants contain from 1 to about 10 amino acid substitutions, deletions, inversions, and/or insertions thereof. Preferably, the Fc region of the Fc fusion molecule and nucleotides encoding the same remain unchanged or are only slightly changed, such as by conservative or neutral amino acid substitution(s).

The Fc fusion molecule preferably disrupts the interaction between MAdCAM and α4β7, and thus reduces, prevents, or eliminates symptoms of a disease state (e.g., MS or related neuroinflammatory conditions).

B. Antibody Therapy

In some embodiments, the present invention provides antibodies that target MAdCAM-α4β7 interaction (e.g., by binding to MAdCAM or β7 integrin) and thus reduce, prevent or eliminate symptoms of a disease state (e.g., MS or related neuroinflammatory conditions). Any suitable antibody (e.g., monoclonal, polyclonal, or synthetic) may be utilized in the therapeutic methods disclosed herein. In preferred embodiments, the antibodies used for therapy are humanized antibodies. Methods for humanizing antibodies are well known in the art (See e.g., U.S. Pat. Nos. 6,180,370, 5,585,089, 6,054,297, and 5,565,332; each of which is herein incorporated by reference).

In some embodiments, the therapeutic antibodies comprise an antibody generated against MAdCAM or β7 integrin, wherein the antibody is conjugated to a cytotoxic agent. For certain applications, it is envisioned that the therapeutic agents will be pharmacologic agents that will serve as useful agents for attachment to antibodies, particularly cytotoxic or otherwise anticellular agents having the ability to kill or suppress the growth or cell division of target cells. The present invention contemplates the use of any pharmacologic agent that can be conjugated to an antibody, and delivered in active form. Exemplary anticellular agents include chemotherapeutic agents, radioisotopes, and cytotoxins. The therapeutic antibodies of the present invention may include a variety of cytotoxic moieties, including but not limited to, radioactive isotopes (e.g., iodine-131, iodine-123, technicium-99m, indium-111, rhenium-188, rhenium-186, gallium-67, copper-67, yttrium-90, iodine-125 or astatine-211), hormones such as a steroid, antimetabolites such as cytosines (e.g., arabinoside, fluorouracil, methotrexate or aminopterin; an anthracycline; mitomycin C), vinca alkaloids (e.g., demecolcine; etoposide; mithramycin), and antitumor alkylating agent such as chlorambucil or melphalan. Other embodiments may include agents such as a coagulant, a cytokine, growth factor, bacterial endotoxin or the lipid A moiety of bacterial endotoxin. For example, in some embodiments, therapeutic agents will include plant-, fungus- or bacteria-derived toxin, such as an A chain toxins, a ribosome inactivating protein, α-sarcin, aspergillin, restrictocin, a ribonuclease, diphtheria toxin or pseudomonas exotoxin, to mention just a few examples. In some preferred embodiments, deglycosylated ricin A chain is utilized.

In any event, it is proposed that agents such as these may, if desired, be successfully conjugated to an antibody, in a manner that will allow their targeting, internalization, release or presentation to blood components at the site of the targeted tumor cells as required using known conjugation technology (See, e.g., Ghose et al., Methods Enzymol., 93:280 [1983]).

For example, in some embodiments the present invention provides immunotoxins targeting MAdCAM or β7 integrin. Immunotoxins are conjugates of a specific targeting agent typically an antibody or fragment, with a cytotoxic agent, such as a toxin moiety. The targeting agent directs the toxin to, and thereby selectively kills, cells carrying the targeted antigen. In some embodiments, therapeutic antibodies employ crosslinkers that provide high in vivo stability (Thorpe et al., Cancer Res., 48:6396 [1988]).

In preferred embodiments, antibody based therapeutics are formulated as pharmaceutical compositions as described below. In preferred embodiments, administration of an antibody composition of the present invention results in a measurable decrease in symptoms of a disease state (e.g., MS or other neuroinflammatory disease).

C. Nucleic Acid Therapeutics

In some embodiments, therapeutics are nucleic acid based (e.g., siRNA or antisense).

1. RNA Interference (RNAi)

In some embodiments, RNAi is utilized to inhibit MAdCAM or MAdCAM binding function or β7 binding function. RNAi represents an evolutionary conserved cellular defense for controlling the expression of foreign genes in most eukaryotes, including humans. RNAi is typically triggered by double-stranded RNA (dsRNA) and causes sequence-specific mRNA degradation of single-stranded target RNAs homologous in response to dsRNA. The mediators of mRNA degradation are small interfering RNA duplexes (siRNAs), which are normally produced from long dsRNA by enzymatic cleavage in the cell. siRNAs are generally approximately twenty-one nucleotides in length (e.g. 21-23 nucleotides in length), and have a base-paired structure characterized by two nucleotide 3′-overhangs. Following the introduction of a small RNA, or RNAi, into the cell, it is believed the sequence is delivered to an enzyme complex called RISC (RNA-induced silencing complex). RISC recognizes the target and cleaves it with an endonuclease. It is noted that if larger RNA sequences are delivered to a cell, RNase III enzyme (Dicer) converts longer dsRNA into 21-23 nt ds siRNA fragments.

Chemically synthesized siRNAs have become powerful reagents for genome-wide analysis of mammalian gene function in cultured somatic cells. Beyond their value for validation of gene function, siRNAs also hold great potential as gene-specific therapeutic agents (Tuschl and Borkhardt, Molecular Intervent. 2002; 2(3):158-67, herein incorporated by reference).

The transfection of siRNAs into animal cells results in the potent, long-lasting post-transcriptional silencing of specific genes (Caplen et al, Proc Natl Acad Sci U.S.A. 2001; 98: 9742-7; Elbashir et al., Nature. 2001; 411:494-8; Elbashir et al., Genes Dev. 2001; 15: 188-200; and Elbashir et al., EMBO J. 2001; 20: 6877-88, all of which are herein incorporated by reference). Methods and compositions for performing RNAi with siRNAs are described, for example, in U.S. Pat. No. 6,506,559, herein incorporated by reference.

siRNAs are extraordinarily effective at lowering the amounts of targeted RNA, and by extension proteins, frequently to undetectable levels. The silencing effect can last several months, and is extraordinarily specific, because one nucleotide mismatch between the target RNA and the central region of the siRNA is frequently sufficient to prevent silencing (Brummelkamp et al, Science 2002; 296:550-3; and Holen et al, Nucleic Acids Res. 2002; 30:1757-66, both of which are herein incorporated by reference).

An important factor in the design of siRNAs is the presence of accessible sites for siRNA binding. Bahoia et al., (J. Biol. Chem., 2003; 278: 15991-15997; herein incorporated by reference) describe the use of a type of DNA array called a scanning array to find accessible sites in mRNAs for designing effective siRNAs. These arrays comprise oligonucleotides ranging in size from monomers to a certain maximum, usually Corners, synthesized using a physical barrier (mask) by stepwise addition of each base in the sequence. Thus the arrays represent a full oligonucleotide complement of a region of the target gene. Hybridization of the target mRNA to these arrays provides an exhaustive accessibility profile of this region of the target mRNA. Such data are useful in the design of antisense oligonucleotides (ranging from 7mers to 25mers), where it is important to achieve a compromise between oligonucleotide length and binding affinity, to retain efficacy and target specificity (Sohail et al, Nucleic Acids Res., 2001; 29(10): 2041-2045). Additional methods and concerns for selecting siRNAs are described for example, in WO 05054270, WO05038054A1, WO03070966A2, J Mol. Biol. 2005 May 13; 348(4):883-93, J Mol. Biol. 2005 May 13; 348(4):871-81, and Nucleic Acids Res. 2003 Aug. 1; 31(15):4417-24, each of which is herein incorporated by reference in its entirety. In addition, software (e.g., the MWG online siMAX siRNA design tool) is commercially or publicly available for use in the selection of siRNAs.

2. Antisense

In other embodiments, MAdCAM or β7 integrin expression is modulated using antisense compounds that specifically hybridize with one or more nucleic acids encoding MAdCAMs or β7 integrins of the present invention. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is modulation of the expression of MAdCAMs or β7 integrins of the present invention. In the context of the present invention, “modulation” means either an increase (stimulation) or a decrease (inhibition) in the expression of a gene. For example, expression may be inhibited to potentially prevent autoimmune inflammation.

It is preferred to target specific nucleic acids for antisense. “Targeting” an antisense compound to a particular nucleic acid, in the context of the present invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding a MAdCAM or β7 integrin of the present invention. The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Since the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). Eukaryotic and prokaryotic genes may have two or more alternative start codons, any one of which may be preferentially utilized for translation initiation in a particular cell type or tissue, or under a particular set of conditions. In the context of the present invention, “start codon” and “translation initiation codon” refer to the codon or codons that are used in vivo to initiate translation of an mRNA molecule transcribed from a gene encoding a tumor antigen of the present invention, regardless of the sequence(s) of such codons.

Translation termination codon (or “stop codon”) of a gene may have one of three sequences (i.e., 5′-UAA, 5′-UAG and 5′-UGA; the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which refers to the region between the translation initiation codon and the translation termination codon, is also a region that may be targeted effectively. Other target regions include the 5′ untranslated region (5′ UTR), referring to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′ UTR), referring to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The cap region may also be a preferred target region.

Although some eukaryotic mRNA transcripts are directly translated, many contain one or more regions, known as “introns,” that are excised from a transcript before it is translated. The remaining (and therefore translated) regions are known as “exons” and are spliced together to form a continuous mRNA sequence. mRNA splice sites (i.e., intron-exon junctions) may also be preferred target regions, and are particularly useful in situations where aberrant splicing is implicated in disease, or where an overproduction of a particular mRNA splice product is implicated in disease. It has also been found that introns can also be effective, and therefore preferred, target regions for antisense compounds targeted, for example, to DNA or pre-mRNA.

In some embodiments, target sites for antisense inhibition are identified using commercially available software programs (e.g., Biognostik, Gottingen, Germany; SysArris Software, Bangalore, India; Antisense Research Group, University of Liverpool, Liverpool, England; GeneTrove, Carlsbad, Calif.). In other embodiments, target sites for antisense inhibition are identified using the accessible site method described in PCT Publ. No. WO0198537A2, herein incorporated by reference.

Once one or more target sites have been identified, oligonucleotides are chosen that are sufficiently complementary to the target (i.e., hybridize sufficiently well and with sufficient specificity) to give the desired effect. For example, in preferred embodiments of the present invention, antisense oligonucleotides are targeted to or near the start codon.

In the context of this invention, “hybridization,” with respect to antisense compositions and methods, means hydrogen bonding, which may be Watson-Crick, Hoogsteen or reversed Hoogsteen hydrogen bonding, between complementary nucleoside or nucleotide bases. For example, adenine and thymine are complementary nucleobases that pair through the formation of hydrogen bonds. It is understood that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired (i.e., under physiological conditions in the case of in vivo assays or therapeutic treatment, and in the case of in vitro assays, under conditions in which the assays are performed).

The specificity and sensitivity of antisense is also applied for therapeutic uses. For example, antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides are useful therapeutic modalities that can be configured to be useful in treatment regimes for treatment of cells, tissues, and animals, especially humans.

While antisense oligonucleotides are a preferred form of antisense compound, the present invention comprehends other oligomeric antisense compounds, including but not limited to oligonucleotide mimetics. The antisense compounds in accordance with this invention preferably comprise from about 8 to about 30 nucleobases (i.e., from about 8 to about 30 linked bases), although both longer and shorter sequences may find use with the present invention. Particularly preferred antisense compounds are antisense oligonucleotides, even more preferably those comprising from about 12 to about 25 nucleobases.

D. Small Molecule Therapies

In other embodiments, the present invention provides small molecule inhibitors of MAdCAM expression, activity, or interaction with α4β7. In other embodiments, the present invention provides small molecule inhibitors of β7 integrin expression, activity, or interaction with MAdCAM. In some embodiments, small molecule therapeutics are identified using drug screening methods (e.g., those described herein).

E. Gene Therapy

The present invention contemplates the use of any genetic manipulation for use in modulating the expression of MAdCAM or β7 integrin. Examples of genetic manipulation include, but are not limited to, gene knockout (e.g., removing the MAdCAM or β7 integrin from the chromosome using, for example, recombination), expression of antisense constructs with or without inducible promoters, and the like. Delivery of nucleic acid construct to cells in vitro or in vivo may be conducted using any suitable method. A suitable method is one that introduces the nucleic acid construct into the cell such that the desired event occurs (e.g., expression of an antisense construct). Genetic therapy may also be used to deliver siRNA or other interfering molecules that are expressed in vivo (e.g., upon stimulation by an inducible promoter).

Introduction of molecules carrying genetic information into cells is achieved by any of various methods including, but not limited to, directed injection of naked DNA constructs, bombardment with gold particles loaded with said constructs, and macromolecule mediated gene transfer using, for example, liposomes, biopolymers, and the like. Preferred methods use gene delivery vehicles derived from viruses, including, but not limited to, adenoviruses, retroviruses, vaccinia viruses, and adeno-associated viruses. Because of the higher efficiency as compared to retroviruses, vectors derived from adenoviruses are the preferred gene delivery vehicles for transferring nucleic acid molecules into host cells in vivo. Adenoviral vectors have been shown to provide very efficient in vivo gene transfer. Examples of adenoviral vectors and methods for gene transfer are described in PCT publications WO 00/12738 and WO 00/09675 and U.S. Pat. Appl. Nos. 6,033,908, 6,019,978, 6,001,557, 5,994,132, 5,994,128, 5,994,106, 5,981,225, 5,885,808, 5,872,154, 5,830,730, and 5,824,544, each of which is herein incorporated by reference in its entirety.

Vectors may be administered to subject in a variety of ways. For example, in some embodiments of the present invention, vectors are administered into tumors or tissue associated with tumors using direct injection. In other embodiments, administration is via the blood or lymphatic circulation (See e.g., PCT publication 99/02685 herein incorporated by reference in its entirety). Exemplary dose levels of adenoviral vector are preferably 10⁸ to 10¹¹ vector particles added to the perfusate.

### II. Therapeutic Applications

As described above, embodiments of the present invention provide compositions and method for treating neuroinflammatory diseases (e.g., MS) associated with aberrant MAdCAM expression on CNS vasculature.

A. Therapeutic Methods

Embodiments of the present invention provide methods of treating neuroinflammatory conditions. Examples of neuroinflammatory conditions that can be treated by the compositions and methods described herein include, but are not limited to, multiple sclerosis (MS), acute trauma (e.g., head or spinal cord injury), Alzheimer's disease, amyotrophic lateral sclerosis (ALS, also known as Lou Gehrig's disease), acute disseminated encephalomyelitis, Bell's palsy, neurosarcoidosis, CNS complications of collagen vascular diseases (including systemic lupus erythematosus, Sjogren's disease, takayasu arteritis, temporal/giant cell arteritis, Wegener's granulomatosis and poyarteritis nodosa), primary CNS angiitis, transverse myelitis, Susac's syndrome, PANDAS (pediatric autoimmune neuropsychiatric disorders associated with streptococcal infections), sydenham's chorea, adrenomyeloneuropathy, Guillain-Barre syndrome, chronic inflammatory demyelinating polyneuropathy, polymyositis, neurobehcet's disease, paraneoplastic syndromes, limbic encephalitis, Lambert-Eaton myasthenic syndrome and myasthenia gravis. Additional neuroinflammatory conditions are within the scope of one of skill in the art.

In some embodiments, the therapeutic compositions described herein are administered to a subject diagnosed or having symptoms of one of the above named neuroinflammatory conditions. In some embodiments, the compositions reduce or eliminate symptoms of the neuroinflammatory conditions. In some embodiments, the compositions reduce the risk of future disease exacerbations. In some embodiments, the compositions reduce the risk of future lesion development as documented on serial radiological scans (e.g., MRI scans). In some embodiments, the compositions accelerate or enhance recovery of active disease. In some embodiments, compositions are administered during episodes of acute or active disease symptoms and therapy is reduced or discontinued when disease symptoms are reduced or eliminated.

In some embodiments, compositions are given to a subject diagnosed with a neuroinflammatory condition but not exhibiting active disease symptoms (e.g., during the remission phase of a relapsing disease) in order to prevent future symptoms. For example, in some embodiments, subjects are administered the described therapeutic compositions as a long term maintenance therapy (e.g., for the remainder of their lives).

In some embodiments, MAdCAM targeted therapeutics are administered in combination with existing therapeutics. In the case of MS, such therapies include, but are not limited to, interferonβ and glatiramer acetate. Treatments for other neuroinflammatory conditions are known to those of ordinary skill in the art and include, but are not limited to, anti-inflammatory agents (e.g., non-steroidal anti-inflammatory agents and steroids), etc.

B. Pharmaceutical Compositions

The present invention further provides pharmaceutical compositions (e.g., comprising pharmaceutical agents that modulate the expression or activity of MAdCAM or β7 integrin). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC50s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the oligonucleotide is administered in maintenance doses, ranging from 0.01 μg to 100 g per kg of body weight, once or more daily, to once every week, month, etc.

### III. Drug Screening Applications

In some embodiments, the present invention provides drug screening assays (e.g., to screen for drugs useful in inhibiting MAdCAM or β7 integrin function or in inhibiting interactions between MAdCAM and β7 integrin). For example, in some embodiments, the present invention provides methods of screening for compounds that alter (e.g., decrease) the expression of MAdCAM or β7 integrin. The compounds or agents may interfere with transcription, by interacting, for example, with the promoter region. The compounds or agents may interfere with mRNA produced from MAdCAM or 137 integrin (e.g., by RNA interference, antisense technologies, etc.). The compounds or agents may interfere with pathways that are upstream or downstream of the biological activity of MAdCAM or β7 integrin. In some embodiments, candidate compounds are antisense or interfering RNA agents (e.g., oligonucleotides) directed against MAdCAM. In other embodiments, candidate compounds are antibodies or small molecules that specifically bind to a MAdCAM regulator or expression products of the present invention and inhibit its biological function. In some embodiments, the drug screening methods described herein identify compounds that block MadCAM binding to binding partners (e.g., α4β7).

In one screening method, candidate compounds are evaluated for their ability to alter MAdCAM expression by contacting a compound with a cell expressing MAdCAM and then assaying for the effect of the candidate compounds on expression. In some embodiments, the effect of candidate compounds on expression of a MAdCAM gene is assayed for by detecting the level of MAdCAM mRNA expressed by the cell. mRNA expression can be detected by any suitable method. In other embodiments, the effect of candidate compounds on expression of MAdCAM genes is assayed by measuring the level of polypeptide encoded by the MAdCAMs. The level of polypeptide expressed can be measured using any suitable method, including but not limited to, those disclosed herein.

Specifically, the present invention provides screening methods for identifying modulators, i.e., candidate or test compounds or agents (e.g., proteins, peptides, peptidomimetics, peptoids, small molecules or other drugs) which bind to MAdCAM or β7 integrin, have an inhibitory (or stimulatory) effect on, for example, MAdCAM expression or MAdCAM activity or β7 integrin expression or activity. Compounds thus identified can be used to modulate the activity of target gene products either directly or indirectly in a therapeutic protocol, to elaborate the biological function of the target gene product, or to identify compounds that disrupt normal target gene interactions. Compounds that inhibit the activity or expression of MAdCAMs are useful in the treatment of neuroinflammatory disease (e.g., MS).

In one embodiment, the invention provides assays for screening candidate or test compounds that interfere with the binding or affinity of MAdCAM protein or polypeptide or a biologically active portion thereof to its ligand. In another embodiment, the invention provides assays for screening candidate or test compounds that bind to or modulate the activity of MAdCAM protein or polypeptide or a biologically active portion thereof. In another embodiment, the invention provides assays for screening candidate or test compounds that bind to or modulate the activity of β7 integrin protein or polypeptide or a biologically active portion thereof.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckermann et al., J. Med. Chem. 37: 2678-85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 [1993]; Erb et al., Proc. Nad. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

Libraries of compounds may be presented in solution (e.g., Houghten, Biotechniques 13:412-421 [1992]), or on beads (Lam, Nature 354:82-84 [1991]), chips (Fodor, Nature 364:555-556 [1993]), bacteria or spores (U.S. Pat. No. 5,223,409; herein incorporated by reference), plasmids (Cull et al., Proc. Nad. Acad. Sci. USA 89:18651869 [1992]) or on phage (Scott and Smith, Science 249:386-390 [1990]; Devlin Science 249:404-406 [1990]; Cwirla et al., Proc. Natl. Acad. Sci. 87:6378-6382 [1990]; Felici, J. Mol. Biol. 222:301 [1991]).

In one embodiment, an assay is a cell-based assay in which a cell that expresses a MAdCAM mRNA or protein or biologically active portion thereof is contacted with a test compound, and the ability of the test compound to the modulate MAdCAM's activity is determined. In one embodiment, an assay is a cell-based assay in which a cell that expresses a β7 integrin mRNA or protein or biologically active portion thereof is contacted with a test compound, and the ability of the test compound to the modulate β7 integrin's activity is determined. Determining the ability of the test compound to modulate MAdCAM or β7 integrin activity can be accomplished by monitoring, for example, changes in binding affinity, destruction or mRNA, or the like.

The ability of the test compound to modulate MAdCAM binding to a compound, e.g., a MAdCAM substrate or modulator, can also be evaluated. This can be accomplished, for example, by coupling the compound, e.g., the substrate, with a radioisotope or enzymatic label such that binding of the compound, e.g., the substrate, to a MAdCAM can be determined by detecting the labeled compound, e.g., substrate, in a complex.

Alternatively, the MAdCAM is coupled with a radioisotope or enzymatic label to monitor the ability of a test compound to modulate MAdCAM binding to a MAdCAM substrate in a complex. For example, compounds (e.g., substrates) can be labeled with ¹²⁵I, ³⁵S, ¹⁴C or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., a MAdCAM ligand) to interact with MAdCAM with or without the labeling of any of the interactants can be evaluated. For example, a microphysiometer can be used to detect the interaction of a compound with a MAdCAM without the labeling of either the compound or MAdCAM (McConnell et al. Science 257:1906-1912 [1992]). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and MAdCAM. In yet another embodiment, a cell-free assay is provided in which a MAdCAM protein or biologically active portion thereof is contacted with a test compound and the ability of the test compound to bind to the MAdCAM protein, mRNA, or biologically active portion thereof is evaluated. Preferred biologically active portions of the MAdCAM proteins or mRNA to be used in assays of the present invention include fragments that participate in interactions with substrates or other proteins, e.g., fragments with high surface probability scores.

Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FRET) (see, for example, Lakowicz et al., U.S. Pat. No. 5,631,169; Stavrianopoulos et al., U.S. Pat. No. 4,968,103; each of which is herein incorporated by reference). A fluorophore label is selected such that a first donor molecule's emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label should be maximal. A FRET binding event can be conveniently measured through standard fluorometric detection means well known in the art (e.g., using a fluorimeter).

In another embodiment, determining the ability of the MAdCAM protein or mRNA to bind to a target molecule can be accomplished using real-time Biomolecular Interaction Analysis (BIA) (see, e.g., Sjolander and Urbaniczky, Anal. Chem. 63:2338-2345 [1991] and Szabo et al. Curr. Opin. Struct. Biol. 5:699-705 [1995]). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

In one embodiment, the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It may be desirable to immobilize MAdCAMs, an anti-MAdCAM antibody or β7 integrin to facilitate separation of complexed from non-complexed forms of one or both of the proteins, as well as to accommodate automation of the assay. Binding of a test compound to a MAdCAM protein, or interaction of a MAdCAM protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase-MAdCAM fusion proteins or glutathione-S-transferase/target fusion proteins can be adsorbed onto glutathione Sepharose beads (Sigma Chemical, St. Louis, Mo.) or glutathione-derivatized microtiter plates, which are then combined with the test compound or the test compound and either the non-adsorbed target protein or MAdCAM protein, and the mixture incubated under conditions conducive for complex formation (e.g., at physiological conditions for salt and pH). Following incubation, the beads or microtiter plate wells are washed to remove any unbound components, the matrix immobilized in the case of beads, complex determined either directly or indirectly, for example, as described above.

Alternatively, the complexes can be dissociated from the matrix, and the level of MAdCAMs binding or activity determined using standard techniques. Other techniques for immobilizing either MAdCAMs protein or a target molecule on matrices include using conjugation of biotin and streptavidin. Biotinylated MAdCAM protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-IgG antibody).

This assay is performed utilizing antibodies reactive with MAdCAM protein or ligand but which do not interfere with binding of the MAdCAMs protein to its ligand. Such antibodies can be derivatized to the wells of the plate, and unbound target or MAdCAMs protein trapped in the wells by antibody conjugation. Methods for detecting such complexes, in addition to those described above for the GST-immobilized complexes, include immunodetection of complexes using antibodies reactive with the MAdCAM protein or target molecule, as well as enzyme-linked assays which rely on detecting an enzymatic activity associated with the MAdCAM protein or target molecule.

Alternatively, cell free assays can be conducted in a liquid phase. In such an assay, the reaction products are separated from unreacted components, by any of a number of standard techniques, including, but not limited to: differential centrifugation (see, for example, Rivas and Minton, Trends Biochem Sci 18:284-7 [1993]); chromatography (gel filtration chromatography, ion-exchange chromatography); electrophoresis (see, e.g., Ausubel et al., eds. Current Protocols in Molecular Biology 1999, J. Wiley: New York.); and immunoprecipitation (see, for example, Ausubel et al., eds. Current Protocols in Molecular Biology 1999, J. Wiley: New York). Such resins and chromatographic techniques are known to one skilled in the art (See e.g., Heegaard J. Mol. Recognit. 11:141-8 [1998]; Hageand Tweed J. Chromatogr. Biomed. Sci. Appl 699:499-525 [1997]). Further, fluorescence energy transfer may also be conveniently utilized, as described herein, to detect binding without further purification of the complex from solution.

The assay can include contacting the MAdCAM protein, mRNA, or biologically active portion thereof with a known compound that binds the MAdCAM to form an assay mixture, contacting the assay mixture with a test compound, and determining the ability of the test compound to interact with a MAdCAM protein or mRNA, wherein determining the ability of the test compound to interact with a MAdCAM protein or mRNA includes determining the ability of the test compound to preferentially bind to MAdCAM or biologically active portion thereof, or to modulate the activity of a target molecule, as compared to the known compound.

To the extent that MAdCAM can, in vivo, interact with one or more cellular or extracellular macromolecules, such as proteins, inhibitors of such an interaction are useful. A homogeneous assay can be used can be used to identify inhibitors. For example, a preformed complex of the target gene product and the interactive cellular or extracellular binding partner product is prepared such that either the target gene products or their binding partners are labeled, but the signal generated by the label is quenched due to complex formation (see, e.g., U.S. Pat. No. 4,109,496, herein incorporated by reference, that utilizes this approach for immunoassays). The addition of a test substance that competes with and displaces one of the species from the preformed complex will result in the generation of a signal above background. In this way, test substances that disrupt target gene product-binding partner interaction can be identified. Alternatively, MAdCAMs protein can be used as a “bait protein” in a two-hybrid assay or three-hybrid assay (see, e.g., U.S. Pat. No. 5,283,317; Zervos et al., Cell 72:223-232 [1993]; Madura et al., J. Biol. Chem. 268.12046-12054 [1993]; Bartel et al., Biotechniques 14:920-924 [1993]; Iwabuchi et al., Oncogene 8:1693-1696 [1993]; and Brent WO 94/10300; each of which is herein incorporated by reference), to identify other proteins, that bind to or interact with MAdCAMs (“MAdCAM-binding proteins” or “MAdCAM-bp”) and are involved in MAdCAM activity. Such MAdCAM-bps can be activators or inhibitors of signals by the MAdCAM proteins or targets as, for example, downstream elements of a MAdCAMs-mediated signaling pathway.

Modulators of MAdCAMs expression can also be identified. For example, a cell or cell free mixture is contacted with a candidate compound and the expression of MAdCAM mRNA or protein evaluated relative to the level of expression of MAdCAM mRNA or protein in the absence of the candidate compound. When expression of MAdCAM mRNA or protein is greater in the presence of the candidate compound than in its absence, the candidate compound is identified as a stimulator of MAdCAM mRNA or protein expression. Alternatively, when expression of MAdCAM mRNA or protein is less (i.e., statistically significantly less) in the presence of the candidate compound than in its absence, the candidate compound is identified as an inhibitor of MAdCAM mRNA or protein expression. The level of MAdCAMs mRNA or protein expression can be determined by methods described herein for detecting MAdCAMs mRNA or protein. A modulating agent can be identified using a cell-based or a cell free assay, and the ability of the agent to modulate the activity of a MAdCAMs protein can be confirmed in vivo, e.g., in an animal such as an animal model for a disease (e.g., an animal with neuroinflammtory disease such as MS).

This invention further pertains to novel agents identified by the above-described screening assays (See e.g., above description of therapeutic agents). Accordingly, it is within the scope of this invention to further use an agent identified as described herein (e.g., a MAdCAM modulating agent, an antisense MAdCAM nucleic acid molecule, a siRNA molecule, a MAdCAM specific antibody, or a MAdCAM-binding partner) in an appropriate animal model (such as those described herein) to determine the efficacy, toxicity, side effects, or mechanism of action, of treatment with such an agent. Furthermore, novel agents identified by the above-described screening assays can be, e.g., used for treatments as described herein.

### IV. Antibodies

The present invention provides isolated antibodies. In some embodiments, the present invention provides monoclonal antibodies that specifically bind to an isolated polypeptide comprised of at least five amino acid residues of MAdCAM or β7 integrin. These antibodies find use in the therapeutic and drug screening methods described herein.

An antibody against a protein of the present invention may be any monoclonal or polyclonal antibody, as long as it can recognize the protein. Antibodies can be produced by using a protein of the present invention as the antigen according to a conventional antibody or antiserum preparation process.

The present invention contemplates the use of both monoclonal and polyclonal antibodies. Any suitable method may be used to generate the antibodies used in the methods and compositions of the present invention, including but not limited to, those disclosed herein. For example, for preparation of a monoclonal antibody, protein, as such, or together with a suitable carrier or diluent is administered to an animal (e.g., a mammal) under conditions that permit the production of antibodies. For enhancing the antibody production capability, complete or incomplete Freund's adjuvant may be administered. Normally, the protein is administered once every 2 weeks to 6 weeks, in total, about 2 times to about 10 times. Animals suitable for use in such methods include, but are not limited to, primates, rabbits, dogs, guinea pigs, mice, rats, sheep, goats, etc.

For preparing monoclonal antibody-producing cells, an individual animal whose antibody titer has been confirmed (e.g., a mouse) is selected, and 2 days to 5 days after the final immunization, its spleen or lymph node is harvested and antibody-producing cells contained therein are fused with myeloma cells to prepare the desired monoclonal antibody producer hybridoma. Measurement of the antibody titer in antiserum can be carried out, for example, by reacting the labeled protein, as described hereinafter and antiserum and then measuring the activity of the labeling agent bound to the antibody. The cell fusion can be carried out according to known methods, for example, the method described by Koehler and Milstein (Nature 256:495 [1975]). As a fusion promoter, for example, polyethylene glycol (PEG) or Sendai virus (HVJ), preferably PEG is used.

Examples of myeloma cells include NS-1, P3U1, SP2/0, AP-1 and the like. The proportion of the number of antibody producer cells (spleen cells) and the number of myeloma cells to be used is preferably about 1:1 to about 20:1. PEG (preferably PEG 1000-PEG 6000) is preferably added in concentration of about 10% to about 80%. Cell fusion can be carried out efficiently by incubating a mixture of both cells at about 20° C. to about 40° C., preferably about 30° C. to about 37° C. for about 1 minute to 10 minutes.

Various methods may be used for screening for a hybridoma producing the antibody (e.g., against a tumor antigen or autoantibody of the present invention). For example, where a supernatant of the hybridoma is added to a solid phase (e.g., microplate) to which antibody is adsorbed directly or together with a carrier and then an anti-immunoglobulin antibody (if mouse cells are used in cell fusion, anti-mouse immunoglobulin antibody is used) or Protein A labeled with a radioactive substance or an enzyme is added to detect the monoclonal antibody against the protein bound to the solid phase. Alternately, a supernatant of the hybridoma is added to a solid phase to which an anti-immunoglobulin antibody or Protein A is adsorbed and then the protein labeled with a radioactive substance or an enzyme is added to detect the monoclonal antibody against the protein bound to the solid phase.

Selection of the monoclonal antibody can be carried out according to any known method or its modification. Normally, a medium for animal cells to which HAT (hypoxanthine, aminopterin, thymidine) are added is employed. Any selection and growth medium can be employed as long as the hybridoma can grow. For example, RPMI 1640 medium containing 1% to 20%, preferably 10% to 20% fetal bovine serum, GIT medium containing 1% to 10% fetal bovine serum, a serum free medium for cultivation of a hybridoma (SFM-101, Nissui Seiyaku) and the like can be used. Normally, the cultivation is carried out at 20° C. to 40° C., preferably 37° C. for about 5 days to 3 weeks, preferably 1 week to 2 weeks under about 5% CO₂ gas. The antibody titer of the supernatant of a hybridoma culture can be measured according to the same manner as described above with respect to the antibody titer of the anti-protein in the antiserum.

Separation and purification of a monoclonal antibody (e.g., against a MAdCAM of the present invention) can be carried out according to the same manner as those of conventional polyclonal antibodies such as separation and purification of immunoglobulins, for example, salting-out, alcoholic precipitation, isoelectric point precipitation, electrophoresis, adsorption and desorption with ion exchangers (e.g., DEAE), ultracentrifugation, gel filtration, or a specific purification method wherein only an antibody is collected with an active adsorbent such as an antigen-binding solid phase, Protein A or Protein G and dissociating the binding to obtain the antibody.

Polyclonal antibodies may be prepared by any known method or modifications of these methods including obtaining antibodies from patients. For example, a complex of an immunogen (an antigen against the protein) and a carrier protein is prepared and an animal is immunized by the complex according to the same manner as that described with respect to the above monoclonal antibody preparation. A material containing the antibody against is recovered from the immunized animal and the antibody is separated and purified.

As to the complex of the immunogen and the carrier protein to be used for immunization of an animal, any carrier protein and any mixing proportion of the carrier and a hapten can be employed as long as an antibody against the hapten, which is crosslinked on the carrier and used for immunization, is produced efficiently. For example, bovine serum albumin, bovine cycloglobulin, keyhole limpet hemocyanin, etc. may be coupled to an hapten in a weight ratio of about 0.1 part to about 20 parts, preferably, about 1 part to about 5 parts per 1 part of the hapten.

In addition, various condensing agents can be used for coupling of a hapten and a carrier. For example, glutaraldehyde, carbodiimide, maleimide activated ester, activated ester reagents containing thiol group or dithiopyridyl group, and the like find use with the present invention. The condensation product as such or together with a suitable carrier or diluent is administered to a site of an animal that permits the antibody production. For enhancing the antibody production capability, complete or incomplete Freund's adjuvant may be administered. Normally, the protein is administered once every 2 weeks to 6 weeks, in total, about 3 times to about 10 times.

The polyclonal antibody is recovered from blood, ascites and the like, of an animal immunized by the above method. The antibody titer in the antiserum can be measured according to the same manner as that described above with respect to the supernatant of the hybridoma culture. Separation and purification of the antibody can be carried out according to the same separation and purification method of immunoglobulin as that described with respect to the above monoclonal antibody.

The protein used herein as the immunogen is not limited to any particular type of immunogen. For example, a MAdCAM or β7 integrin of the present invention (further including a gene having a nucleotide sequence partly altered) can be used as the immunogen. Further, fragments of the protein may be used. Fragments may be obtained by any methods including, but not limited to expressing a fragment of the gene, enzymatic processing of the protein, chemical synthesis, and the like.

### EXPERIMENTAL

The following examples are provided in order to demonstrate and further illustrate certain preferred embodiments and aspects of the present invention and are not to be construed as limiting the scope thereof.

### Example 1

### Expression of α4β7 on Encephalitogenic T cells and MAdCAM-1 on CNS Blood Vessels

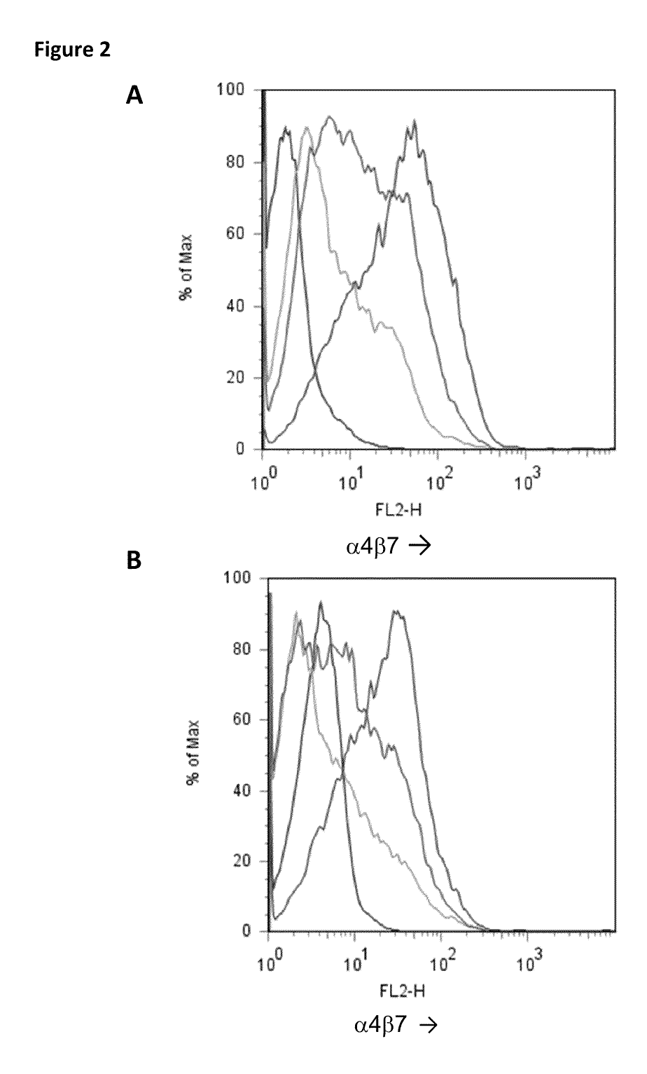

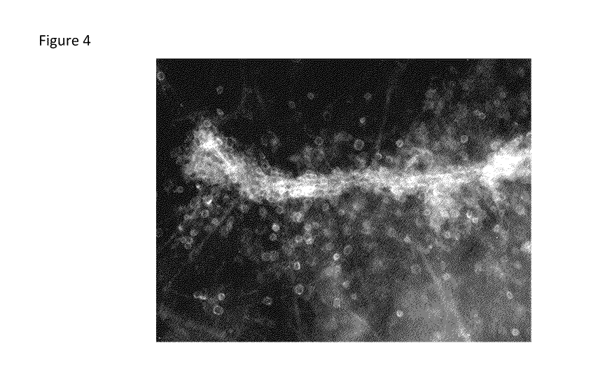

Although α4β7 is typically characterized as a marker of gut homing T cells, it is preferentially expressed on myelin-specific Th17 cells, and to a lesser extent on Th1 cells, that traffic to the CNS and accumulate in EAE lesions (FIG. 2, 3). Furthermore, MAdCAM-1, the high affinity ligand of α4β7, is upregulated on inflamed CNS vessels in mice with acute EAE (FIG. 4). The latter finding is corroborated by four previous publications that demonstrated expression of MAdCAM-1 on brain endothelial cells in the setting of neuroinflammation.

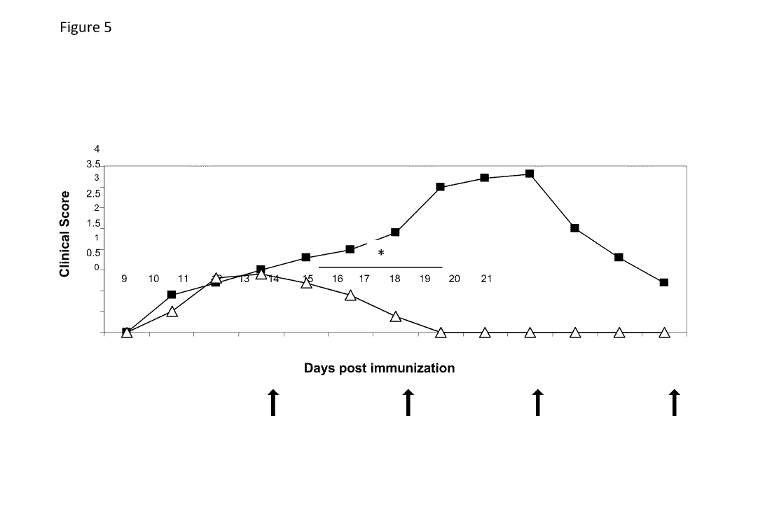

Blockade of α4β7/MAdCAM-1 interactions as a therapy for autoimmune demyelinating disease. In order to assess the importance of α4β7/MAdCAM-1 interactions for the clinical manifestation of EAE, a monoclonal antibody specific for MAdCAM-1 or an isotype matched control antibody to PLP₁₃₉₋₁₅₁₋immunized SJL mice is administered several days after the onset of neurological deficits. As shown in FIG. 5, the mice treated with anti-MAdCAM-1 experienced an accelerated and more complete recovery than mice treated with control antibody.

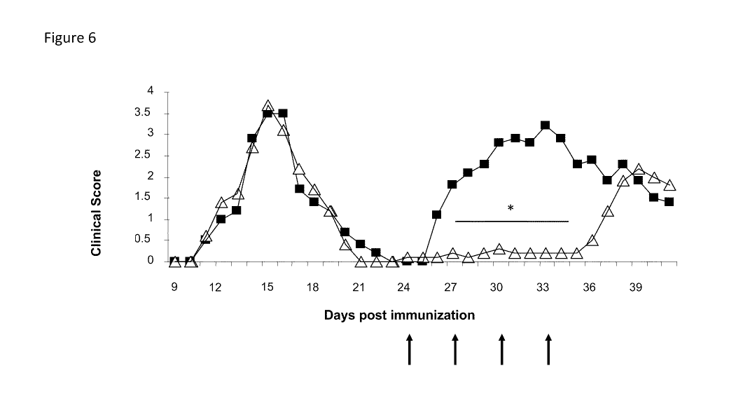

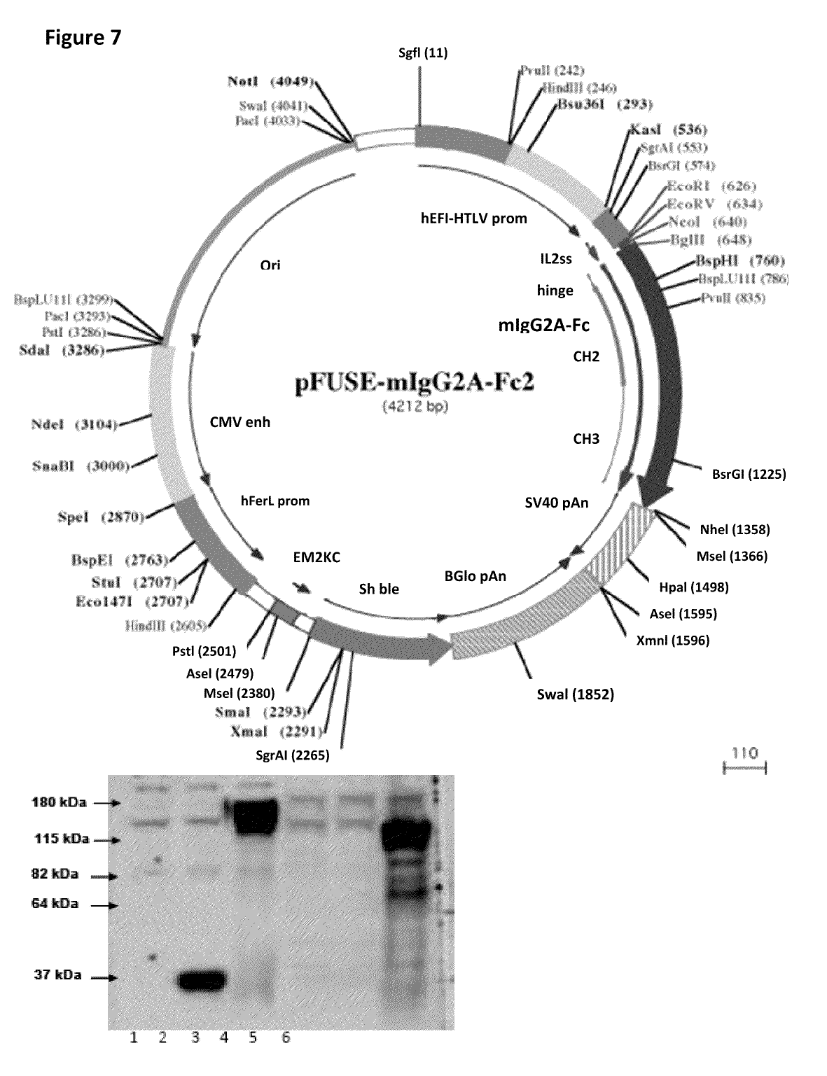

The above experiment simulates the clinical scenario when patients begin treatment in the midst of their first clinically manifested episode of autoimmune demyelinating disease (i.e., at the time of presentation of a “clinically isolated syndrome.”) In order to simulate the treatment of patients with definite multiple sclerosis, we delayed treatment with anti-MAdCAM-1 mAbs until the remission phase of relapsing EAE. When administered following recovery from the presenting episode, anti-MAdCAM-1 completely suppressed a subsequent clinical relapse. The therapeutic effect persisted through the course of antibody administration. Mice began to succumb to clinical EAE shortly after the therapy was withdrawn (FIG. 6).

Construction of a MAdCAM-1-Fc fusion protein. The above experiments indicate that blockade of MAdCAM-1 dependent interactions suppresses established autoimmune demyelinating disease. In order to develop a reagent that would specifically inhibit interactions between activated α4β7⁺ T cells and inflamed blood vessels in the CNS, we constructed a MAdCAM-1-Fc fusion protein using the plasmid, pFUSE-mIgG2A-Fc2 (InvivoGen). This plasmid contains genes encoding the IL-2 signal sequence (IL-2ss, to facilitate secretion of the Fc-Fusion proteins), the Fc region of mouse IgG2A heavy chain (CH2 and CH3 domains and the hinge region), and a multiple cloning site (FIG. 7A). The extracellular domain of MAdCAM-1 was PCR amplified from cDNA derived from NSF-60 cells using primers with the following sequences: 5′ GAC GAA TTC GCA GTC CTT CCA GGT GAA CCC C₃′ (forward); 5′ GAC AGA TCT ATT CGG GGT CAC CTG GCC 3′ (reverse). The sequence of the amplified product was confirmed by the University of Michigan Sequencing Core Facility. Amplified MAdCAM-1 fragments were then inserted in the pFUSE-mIgG2A-Fc2 plasmid using EcoRI NcoI restriction sites located between the IL-2ss and Fc genes. Recombined plasmids were transfected into human embryonic kidney (HEK) 293T cells and selected with Zeocin. The presence of fusion protein in the conditioned media and cell lysates of transfectants was confirmed by Western blot analysis (FIG. 7B). A large quantity of fusion protein was purified from supernatants by protein A affinity chromotography.

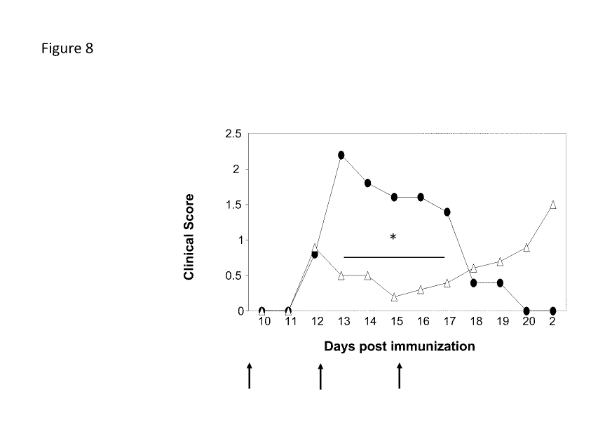

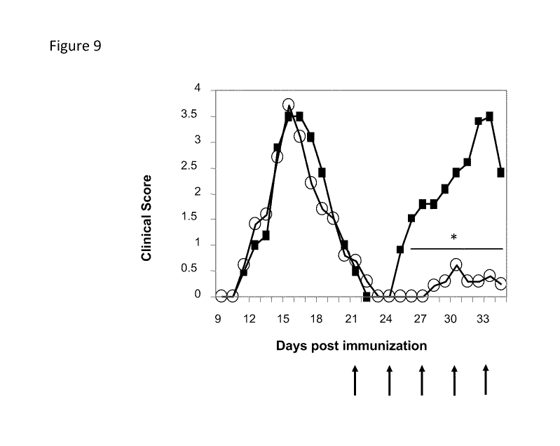

Treatment of mice with MAdCAM-1-Fc fusion protein during different stages of EAE. PLP139-151 immunized SJL mice were treated with MAdCAM-1-Fc fusion protein during the first episode (FIG. 8) or during the first remission of relapsing EAE (FIG. 9). Clinically comparable groups were treated with a control (unfused) Fc protein in parallel. MAdCAM-1-Fc was therapeutically effective in both instances. Consistent with findings with the anti-MAdCAM-1 mononclonal antibody, mice began to develop neurological signs within 1-2 days of withdrawing MAdCAM-1-Fc treatments (FIG. 8).

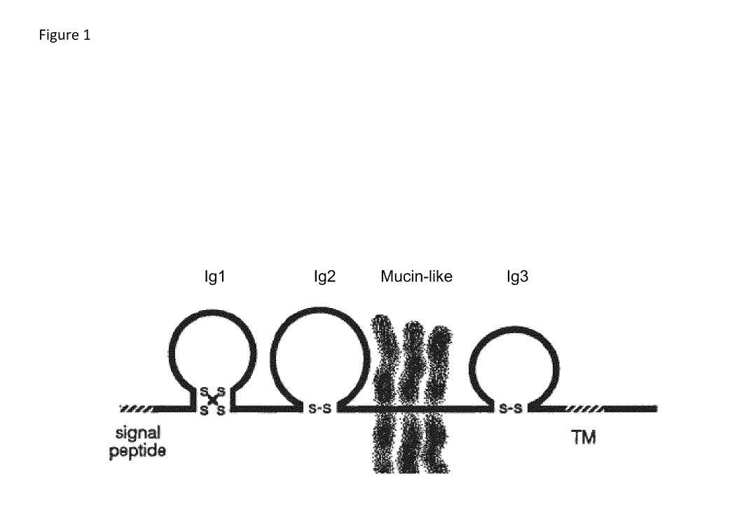

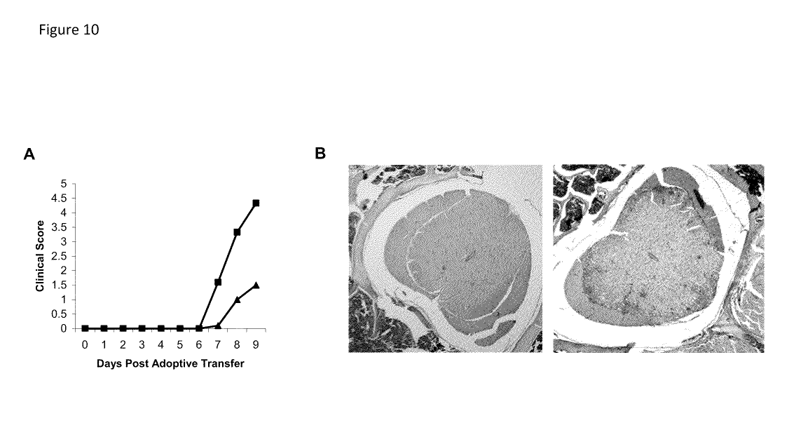

MAdCAM-1-Fc treatment inhibits EAE induced by the adoptive transfer of myelin-specific Th17 cells. The data in FIG. 1 indicate that MAdCAM-1 blockade is effective in preventing clinical EAE induced by myelin-specific effector cells that have been differentiated along the Th17 lineage. To investigate that possibility, naive SJL mice were injected with PLP₁₃₉₋₁₅₁ specific CD4⁺ Th17 cell lines and treated them with either anti-MAdCAM-1 (MECA 89) or an isotype matched control mAb. Mice treated with anti-MAdCAM-1 experienced a delayed and relatively mild course of EAE compared to mice treated with control mAb (FIG. 10A). Analysis of spinal cords from both treatment groups revealed that anti-MAdCAM-1 treatment resulted in less CNS inflammation and demyelination (FIG. 10B).

### Example 2

### Expression of α4β7⁺ on CNS Infiltrating CD4⁺ T Cells and MAdCAM-1 on CNS Endothelium During the Course of Relapsing and Chronic EAE

The extravasation of leukocytes into the CNS parenchyma from the bloodstream is a critical event in the formation of EAE and MS lesions. This is a multi-step process, initially involving selectin-mediated “rolling” of circulating T cells as they traverse postcapillary venules, followed by their firm adhesion to the vascular lining, which is mediated by integrins. As has been observed with chemokine receptors, CD4⁺ T cells express different panels of adhesion molecules based on their Th lineage which, in turn, influences their trafficking patterns. For example, Th1 cells express high levels of P-selectin ligand by comparison to Th2 cells, conferring them with a selective advantage in accessing the peritoneal cavity following intraperitoneal injection of thioglycollate. Experiments conducted during the course of development of the present invention demonstrated that α4β7 is upregulated on myelin-specific Th17 and, to a lesser extent, Th1 cells during polarization in vitro (FIG. 2). Furthermore, MAdCAM-1 is expressed on CNS endothelium at sites of inflammatory inflammation in mice with EAE (FIG. 4).

SJL mice are immunized with PLP₁₃₉₋₁₅₁ in CFA to induce relapsing remitting EAE. Representative mice are sacrificed at defined stages (including the preclinical stage, presenting episode, first remission, first relapse and subsequent relapses and remissions). In parallel experiments, C57BL/6 mice are immunized with an immunodominant epitope of myelin oligodendrocyte glycoprotein (MOG₃₅₋₅₅) in CFA and inject Bordetella toxin (300 ng I.V.) on days 0 and 2 in order to induce chronic EAE. Representative mice are sacrificed during the preclinical stage and early and late progression. Single cell suspensions of CNS mononuclear cells (isolated over a 30%/70% Percoll gradient), splenocytes, mesenteric lymph nodes and pooled draining (brachial and inguinal) lymph nodes from each experimental group are stained with various panels of fluorochrome-conjugated monoclonal antibodies specific for the following markers: activated α4β7 heterodimer, activated α4β7 heterodimer, P-selectin ligand, CD3, CD4, CD62L, CD25, CD44, CCR5 and CXCR3. The level of α4β7, other adhesion molecules and chemokine receptors is then be measured on gated CD4⁺CD3⁺ cells by flow cytometric analysis. In order to determine the cytokine production of α4β7⁺ and α4β7⁻ CD4⁺ T cells, some aliquots are costained intracellularly with antibodies against IFNγ, IL-17, IL-2, IL-4 and/or IL-10.

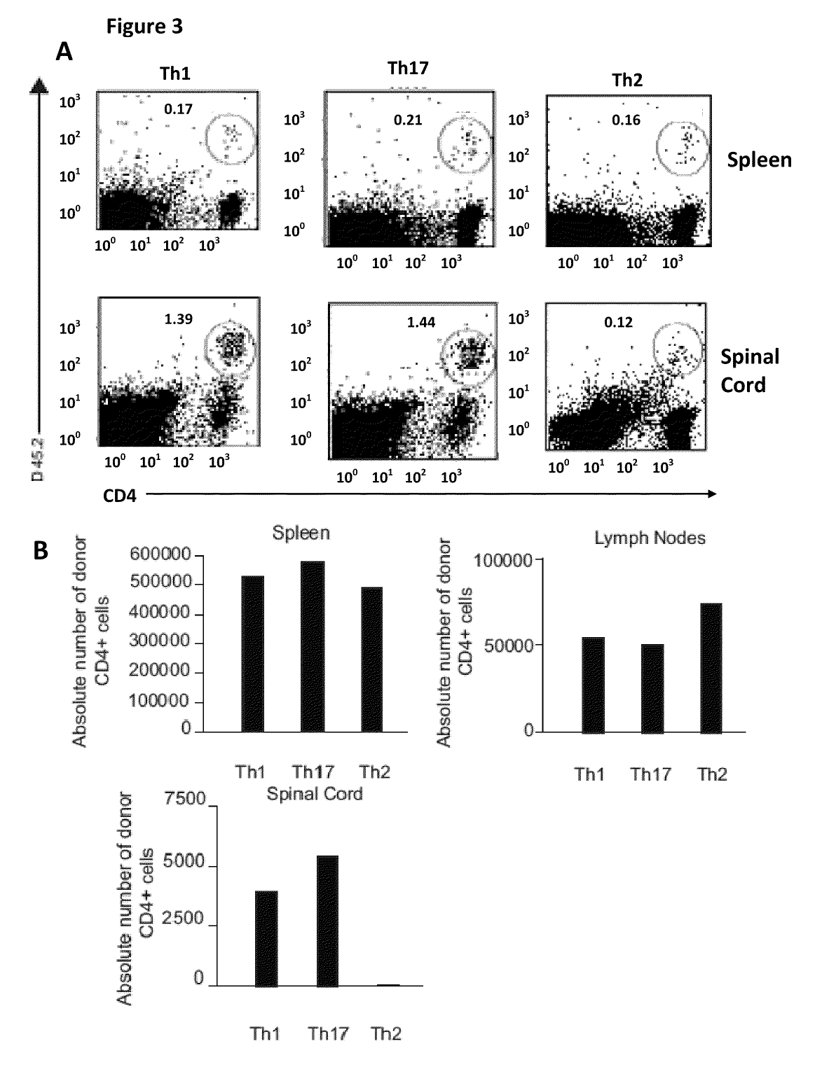

In addition to myelin-specific T cells, a significant number of non-specific lymphocytes are recruited to EAE lesions. In order to specifically determine whether α4β7⁺ cells are enriched among the myelin-specific CD4⁺ Th cell subset in the CNS of mice with EAE, the experimental system described in FIG. 3 is used. CD45.1 congenic CD4⁺ 2D2 cells (that express a transgenic T cell receptor specific for MOG₃₅₋₅₅) are parked in CD45.2⁺ C57BL/6 hosts. One day later the hosts ware actively immunized with MOG peptide. Mice are sacrificed following the clinical onset of EAE and flow cytometric analysis is performed on CNS monocular cells, splenocytes and lymph node cells following staining with anti-CD45.1 and anti-CD45.2 in combination with anti-α4β7 and other mAbs listed above. The expression of α4β7 in addition to other cell surface molecules on donor and host cells is determined by gating on CD45.1⁺CD4⁺ cells or CD45.2⁺CD4⁺ cells, respectively. In some instances, intracellular staining is performed to determine the Th phenotype of α4β7⁺ CD4⁺ T cells. In complementary experiments CNS mononuclear cells isolated from symptomatic C57BL/6 mice or SJL mice with are costained with MOG₃₅₋₅₅- or PLP₁₃₉₋₁₅₁-pulsed MHC Class II tetramers (obtained from V. J. Kuchroo, Harvard Medical School) as well as mAbs against α4β7 and other cell surface molecules and/or cytokines as detailed above.

It is contemplated that a relatively high percentage of CD4⁺ T cells in the CNS of mice with active EAE will be α4β7 positive. Enrichment of α4β7^{hi} cells will be particularly pronounced among myelin-specific Th17 cells within EAE infiltrates. It is possible that CD4⁺ T cells downregulate α4β7 after they enter the CNS. This would result in a relative dearth, rather than excess, of α4β7^{hi} cells within EAE infiltrates. Previous adoptive transfer studies have revealed that labeled myelin-specific T cells accumulate in the cervical lymph nodes prior to their entry into the CNS. Other studies have found that antigens directly introduced into the CNS by intracerebral innoculation are subsequently presented to T cells within the cervical lymph nodes. Therefore, if the percentage α4β7^{hi}CD4⁺ T cells and/or the mean fluorescent intensity of α4β7 is reduced among CNS mononuclear cells, cervical lymph nodes from mice at different stages of EAE are analyzed for the presence of α4β7^{hi} CD4⁺ T cells. It is contemplated that α4β77^{hi} CD4⁺ myelin-specific T cells are enriched in the cervical lymph nodes during the preclinical and acute phases of EAE, irrespective of α4β7 expression on T cells within the CNS at the same time points. In addition, adoptive transfer studies are performed to directly determine whether α4β7 staining is lost on Th17 donor cells after they traverse the blood-brain-barrier. To do so, CFSE-labeled myelin-specific Th17 cells are injected into CD45.1 congenic hosts. α4β7 levels are measured on donor cells prior to transfer, as well as post transfer when they have accumulated in the CNS, cervical lymph nodes and spleen.

As shown in FIG. 2, myelin-specific CD4⁺ T cells upregulate α4β7 during culture under Th17 polarizing conditions. The specific signals responsible for this event remain to be identified. In the mesenteric lymph nodes expression of α4β7 is enhanced on T cells by a retinoic acid dependent mechanism.

FACS sorted naive (CD62L⁺CD44⁻CD25⁻) CD4+ T cells that express a transgenic T cell receptor specific for MOG₃₅₋₅₅ or MBPAc₁₋₁₁ are stimulated with or without antigen and syngenic bone marrow derived dendritic cells or T-depleted splenocytes in the presence of various combinations of recombinant TGF-β, IL-6 and IL-β, or in the absence of exogenous cytokines. In some cases, the retinoic acid dehydrogenase inhibitor citral or the retinoic acid receptor antagonist LE135 is added to culture wells. At serial time points (6, 12, 24, 48 and 72 hours) cells will be harvested and analyzed by flow cytometry to measure the percent of α4β7⁺ cells as well as mean fluorescent intensity of α4β7 on positive cells. It is also determined if IL-23 reinforces and/or enhances α4β7 on Th17 memory cells. Similar experiments are conducted to assess the effects of Th1 polarizing agents (IL-12p70 and IFNγ) and Th2 polarizing agents (IL-4) on α4β7 levels.

It is contemplated that stimulation of naive autoreactive CD4⁺ T cells with antigen and either TGF-β and IL-6 or IL-12p70 will induce α4β7 expression. TGF-β or IL-6 will not induce α4β7 when administered alone. Upregulation of α4β7 will be inhibited by citral and LE135.

Based on the results of the experiments above, CD45.1 congenic IL-12Rβ1 deficient, IL-12Rβ2 deficient, TGF-β receptor1 dominant negative, or wildtype 2D2 cells are introduced into CD45.2+ C57BL/6 hosts and EAE is induced by active immunization with MOG₃₅₋₅₅. Mice are sacrificed during acute EAE and the number of CD45.1⁺ CD4⁺ donor cells that have entered the CNS is determined by flow cytometric analysis. CD45.1⁺ CD4⁺ donor cells within the CNS mononuclear fraction and in peripheral lymphoid tissues are analyzed for expression of active α4β7. It is contemplated that IL-12Rβ1 deficient and TGF-β receptor1 dominant negative CD4⁺ T cells will fail to upregulate α4β7 and accumulate in the CNS. It is also determined if in vivo administration of citral and LE135 to actively immunized mice prevents myelin-specific T cells from upregulating α4β7 and infiltrating the CNS.

As described above, two models of EAE, one that simulates a relapsing-remitting course (SJL mice immunized with PLP₁₃₉₋₁₅₁) and one that simulates a chronic course (C57BL/6 mice immunized with MOG₃₅₋₅₅) are utilized. Spinal cord and brain samples, harvested from representative mice at different stages of EAE, are fixed in 4% paraformaldehyde, embedded in OCT (Tissue-Tek) and flash frozen in liquid nitrogen. Air dried sections are then stained with chromagen labeled antibodies specific for MAdCAM-1, VCAM, ICAM and CD31 (a pan-endothelial cell marker) and imaged under a Olympus BX50 upright light microscope with a MagnaFire digital camera and analyzed with Spot Software Windows version 4.0.4 imaging software. In order to determine whether α4β7⁺ T cells cluster around MAdCAM-1⁺ vessels, perivascular inflammatory cells are stained in contiguous sections with antibodies against CD3, CD4 and α4β7 integrin. Alternatively, contiguous sections are stained with anti-CD3, anti-CD4, and recombinant MAdCAM-1-FITC derived from the same cDNA sequences used for the construction of a MAdCAM-1-Fc chimeric fusion protein. As a negative control, background staining is assessed by incubating tissue sections with a mutant variant of MAdCAM-1-FITC that selectively lacks the α4β7 binding motif.

In a complementary approach, mRNA encoding MAdCAM-1, VCAM, ICAM or NKX2.3 (a transcription factor that directly binds and activates the MAdCAM-1 promoter) is measured in CNS and lymphoid specimens from mice with EAE by real time RT-PCR. Messenger RNA extracted from mice that have been immunized with irrelevant foreign antigens in CFA serves as a negative control. Mesenteric lymph node mRNA serves as a positive control. Adhesion molecule transcript copy numbers are normalized to CD31 mRNA to control for vessel density.

It is contemplated that MAdCAM-1 will be segmentally upregulated on endothelial cells within EAE lesions. α4β7⁺CD4⁺ T cells will tend to cluster around MAdCAM-1⁺ blood vessels. VCAM-1 will be more widely expressed in the vasculature than MAdCAM-1.

If a discrepancy is found in MAdCAM-1 expression between the immunohistochemistry and real-time PCR assays at particular time points, in situ hybridization is utilzed. Samples of spinal cord and brain (as well as mesenteric lymph node and intestine, used as positive controls) are snap frozen in OCT. Cryosections are be prepared and placed on gelatin-coated slides, fixed in 3% paraformaldehyde, rinsed in PBS, and incubated in hybridization buffer (containing denatured salmon sperm DNA) followed by exposure to digoxigenin-labeled MAdCAM-1 antisense probe. After hybridization, slides are washed, blocked with 10% fetal bovine serum and treated with antibodies specific for digoxigenin-conjugated with peroxidase (Boehringer Manheim). For detection of the digoxignenin-labeled probes slides are incubated with substrate DAB (Boehringer Manheim) and viewed under a phase contrast microscope. Negative controls include CNS sections from naive mice incubated with the MAdCAM-1 probe as well as CNS sections from mice with EAE incubated with sense probe. In order to confirm that MAdCAM-1 mRNA is expressed in endothelial cells, sections are costained with FITC-conjugated anti-CD31 mAb and reimaged under a fluorescent microscope. Adhesion molecules, such as VCAM-1, are induced on endothelial cells by soluble factors released by circulating inflammatory cells, such as TNFα and IL-1β. The factors that stimulate CNS endothelial cells to upregulate MAdCAM-1 are unknown. It is contemplated that IL-17 or GM-CSF, released or induced by Th17 cells, are responsible. This is consistent with the finding that MAdCAM-1 mRNA is upregulated in the CNS following the adoptive transfer of Th17 cells, immediately prior to the expected day of onset of EAE. The expression of MAdCAM-1 as well as VCAM-1 and I-CAM in the CNS is monitored on a daily basis following adoptive transfer of myelin-specific T cells with different cytokine profiles. Donor lymph node cells are obtained from MOG₃₅₋₅₅ immunized C57BL/6 mice. They are stimulated with antigen under Th17 polarizing (IL-23, anti-IFNγ, anti-IL-4), Th1 polarizing (IL-12p70, IFNγ, anti-IL-4, anti-IL-23p19) or neutral (no recombinant cytokines or monoclonal antibodies) conditions. After four days, the cells are harvested, washed and injected into naive syngeneic C57BL/6 mice (10⁷ cells/mouse i.p.). The hosts are sacrificed during the preclinical stage and acute EAE for measurement of MAdCAM-1, VCAM-1 and I-CAM in the CNS by real time RT-PCR and immunohistochemistry. NKX2.3 (a MAdCAM-1 associated transcription factor) is also be measured by RT-PCR. If Th17-polarized cells induce MAdCAM-1 expression in the CNS vasculature, these studies are repeated with IL-17 receptor deficient or GM-CSF receptor deficient recipients. If Th1 polarized cells induce CNS MAdCAM-1, the experiments are repeated with IFNγ receptor deficient hosts. It is also determined whether incubation of brain endothelial cell line (hCMEC/D3) with recombinant IL-17, GM-CSF or IFNγ stimulates NKX2.3 and MAdCAM-1 expression.

It is contemplated that MAdCAM-1 will be upregulated on CNS endothelial cells by IL-17 and/or GM-CSF signaling. If Th17 induces CNS MAdCAM-1 independent of IL-17 or GM-CSF signaling, the role of cell-to cell-interactions is investigated. In particular, transwell experiments are performed with activated Th17 cells and brain endothelial cells. It is also determined if blockade of CD40 or LFA-1 in vivo prevents upregulation of MAdCAM-1 following the adoptive transfer of Th17 polarized MOG-specific T cells.

### Example 3

### Therapeutic Consequences of α4β7 Blockade in EAE

The therapeutic efficacy of MAdCAM-1-Fc treatment in EAE when administered at different stages during a relapsing remitting or chronic course is investigated. A panel of control fusion proteins is generated. Hence, for a negative control, an altered variant of MAdCAM-1-Fc that can no longer bind α4β7 is engineered. As discussed earlier, mutation of L40, D41, and T42 in the MAdCAM ectodomain completely abrogates its interaction with α4β7. Therefore, a MAdCAM variant in which these residues are replaced by alanine is generated (henceforth referred to as vMAdCAM-1-Fc).

In addition, an ectodomain deletion mutant that retains the α4β7 binding domains (Ig1 and Ig2) but lacks the mucin-like and IgA1 homologues sequences is generated. The two N-terminal domains of MAdCAM-1 are sufficient to confer α₄β₇ binding comparable to that of native MAdCAM-1. Hence, an ectodomain deletion mutant allows one to determine whether MAdCAM-Fc inhibits EAE by antagonism of the α4β7 integrin receptor alone. The deletion mutant as ΔMAdCAM-\tilde{1}.

Standard molecular biology methods are used to construct vMAdCAM, vMAdCAM-Fc, ΔMAdCAM, and ΔMAdCAM-Fc, as previously described in detail (Robak et al., 2009). Briefly, site directed mutagenesis is carried out using a fusion PCR approach with overlapping primers in which the codons of L40, D41, and T42 in MAdCAM have been replaced by GCC or GCA (codons for alanine). The transmembrane spanning and cytoplasmic portion of MAdCAM is included to obtain membrane bound versions of vMAdCAM and ΔMAdCAM. For soluble proteins, the ectodomain of vMAdCAM or ΔMAdCAM is fused to the constant region of human IgG1, as described previously (Robak et al., 2009). All constructs are verified by DNA sequencing and expression of recombinant proteins is analyzed by Western blotting using anti-MAdCAM-1 antiserum (as shown in FIG. 3b).

It has previously been shown that ectopic expression of full-length MAdCAM in COS cells is sufficient to confer binding of lymphocytes. The ability of T cells to bind COS-7 cells transfected with DNA encoding either wildtype MAdCAM-1, vMAdCAM-1 or ΔMAdCAM-1 is assayed. COS-7 cells are cultured on polylysine (50 microgram/ml) coated 24-well plates prior to transient transfection with lipofectamine 2000. For these assays the T cell lymphoma TK1 cell line, Th17 polarized 2D2 cells or myelin-specific CD4⁺ T cell lines isolated from SJL or C57BL/6 mice with EAE are used. To measure lymphocyte binding, the cells will be labeled with 2,7-bis-(2-carboxyethyl)-5(and-6)-carboxyfluorescein (BCECF: Molecular Probes) for 30 minutes. Cells are then washed for 10 minutes and resuspended in RPMI 1640 supplement with 5 mg/ml BSA, and plated onto transfected COS-7 cells at a density of 2×10⁵ cells per well (in a 24-well plate) for 20 minutes. Non-bound lymphocytes are removed by three rinses in RPMI and bound cells are lysed in 0.1% SDS, 50 mM TrisHCl pH 8.5 for 10 minutes. The released fluorochromophore from lyzed cells are analyzed with a microplate reader. To demonstrate that binding to full-length wild-type MAdCAM and ΔMAdCAM is specific, some assays are carried out in the presence of the anti-MAdCAM mAbs MECA-89 or MECA-367. As a negative control for lymphocyte binding studies, untransfected COS-7 cells, or COS-7 transiently transfected with an eGFP expression construct is used and compared to membrane bound vMAdCAM. Binding of unpolarized myelin-specific lymphocytes to COS-7 cells transiently transfected with wildtype MAdCAM-1 is also assessed. To assess cell surface expression levels of recombinant MAdCAM protein, transiently transfected COS-7 cells are immunolabled with anti-MAdCAM under non-permeabilizing conditions as described previously.

Additional binding studies in the presence of soluble Fc fusion proteins are performed to assess the efficacy of MAdCAM-Fc, ΔMAdCAM-Fc and vMAdCAM-Fc in perturbing the interaction between lymphocytes and transiently transfected COS-7 cells expressing full-length MAdCAM or VCAM-1. Lymphocytes are labeled with BCECF as described above and incubated with increasing concentrations of soluble MAdCAM-Fc fusion proteins (0-100 μg/ml). To mimic activation of the α4 surface integrin receptors, some assays are carried out in the presence of Mn2+ (1 mM).

Leukocytes bearing either α4β7 or the α4β1 integrin receptor are able to bind to endothelial cells expressing VCAM-1, whereas only cells expressing α4β7 are able to bind to endothelium expressing MAdCAM-1. Thus, as a specificity control for the COS cell binding assays, lymphocyte binding to cells transiently transfected with full length VCAM-1 plasmid DNA are used. Lymphocyte binding to VCAM-1 transfected COS-7 cells is carried out in the in the presence or absence of MAdCAM-Fc fusion proteins. Because MAdCAM does not associate with α4β1 integrin, it is expected that the α4β1-VCAM-1 is not perturbed.

To mimic the potential of soluble MAdCAM-Fc fusion protein to block binding of lymphocytes to brain endothelial cells of control or EAE mice, cryosections of unfixed brain tissue are cut and postfixed for 8 minutes in 100% methanol at −20 C degrees. This is a gentle way to fix brain tissue; many ligand receptor interactions remain preserved using this method. Binding and quantification of BCECF labeled lymphocytes to brain tissue sections is carried out as described above. A similar binding assay to detect lymphocyte interactions with MAdCAM expressed in situ has been reported previously to demonstrate binding to inflamed intestine.

PLP₁₃₉₋₁₅₁ immunized SJL mice are treated with MAdCAM-1-Fc, ΔMAdCAM-1-Fc, vMAdCAM-1-Fc or VCAM-1-Fc beginning at the peak of the first clinical episode, during the first remission, at the peak of the first relapse or during subsequent relapses and remissions. Initially, a dosing regimen of 0.5 mg of MAdCAM-1-Fc every three days (FIGS. 5, 6) is utilized. Based on the results of these experiments alternative dosing regimens are tested by using smaller quantities of fusion proteins spaced further apart.

Mice in each group are scored for degree of paralysis on a daily basis using a standard 5 point scale by an examiner blinded to the treatments. Clinical outcome measures are complemented by histological studies of CNS tissues (Slides are stained with hematoxylin and eosin or Luxol Fast Blue to evaluate neuroinflammation and demyelination, respectively) and immunological assays (antigen specific IFNγ and IL-17 Elispot/ELISA responses and proliferation).

In order to simulate the chronic course of MS, C57BL/6 mice are immunized with MOG₃₅₋₅₅ in CFA and injected with Bordetella toxin (300 ng i.v.) on days 0 and 2 post-immunization. At various time points following clinical onset, mice are treated with MAdCAM-1-Fc, ΔMAdCAM-1-Fc, vMAdCAM-1-Fc or VCAM-1-Fc according to a variety of dosing regimens. As above, animals in each group are evaluated by clinical scores, histopathological analyses and immunological assays.

It is contemplated that administration of either wildtype MAdCAM-1-Fc or ΔMAdCAM-1-Fc, but not vMAdCAM-1-Fc, during established EAE will prevent relapses in SJL mice and reduce disability in C57BL/6 mice. These therapeutic effects will persist through the duration of treatment. Wildtype MAdCAM-1-Fc and ΔMAdCAM-1-Fc will be as effective as VCAM-1-Fc in suppressing disease and reducing neuroinflammation and demyelination.

MAdCAM-Fc inhibits EAE relapse when given during the first remission (FIG. 9). This experiment was repeated three times with similar results. If it is found that MAdCAM-Fc loses its efficacy after a certain duration of treatment, or when initiated following several relapses, it is determined whether peripheral CD4+ Th17 and Th1 cells (including myelin-peptide/tetamer positive cells or parked 2D2 cells) downregulate a4137 and/or CNS endothelial cells downregulate MAdCAM-1 as the disease evolves. In addition, it is determined whether peripheral CD4+ T cells and CNS endothelial cells express/upregulate different panels of adhesion molecules over time. Candidate alternative adhesion molecule pairs include P-selectin ligand/P-selectin, αEβ7 integrin/E-cadherin, α5β1/fibronectin, LFA-1/ICAM-1 and LFA-3/1-CAM.

Th1 and Th17 polarized myelin-specific T cells lines are both capable of inducing EAE. Furthermore, Th1 and Th17 induced disease are clinically indistinguishable. However, each pathogenic subset triggers a distinct chemokine and effector molecule profile in the CNS and the two forms of EAE display distinct patterns of responsiveness to specific cytokine inhibition. It was found that myelin-specific Th17 polarized CD4⁺ T cells express high levels of α4β7 compared to other Th subsets (FIG. 2). α4β7 is also upregulated on myelin-specific Th1 cells compared to uncommitted T cells, though to a lesser extent than on Th17 cells. Collectively these observations suggest that Th17 induced EAE might be more dependent on α4β7-MAdCAM-1 interactions than Th1 induced EAE.

To investigate that possibility PLP-specific Th1 or Th17 CD4⁺ T cells are adoptively transferred into naive syngeneic hosts and the recipients are treated with MAdCAM-1-Fc, ΔMAdCAM-1-Fc, or vMAdCAM-1. In parallel experiments adoptive transfer recipients are treated with VCAM-1-Fc or an appropriate control fusion protein as a control. Donor cells are derived from SJL mice immunized with PLP₁₃₉₋₁₅₁ emulsified in IFA as previously described. Draining lymph node cells are cultured under Th17 polarizing (IL-23, anti-IFNγ and anti-IL-4) or Th1 polarizing (IL-12p70, IFNγ, anti-IL-4, anti-IL-23p19) conditions. After a 4 day incubation period, cells are harvested, washed and injected into naive SJL mice (10⁷ cells/mouse i.p.). Host mice in each group are treated with recombinant fusion proteins from the day of transfer onward (500 μg/mouse i.v. every third day) and evaluated on a daily basis for signs of neurological dysfunction. Representative mice from each group are sacrificed for histopathological studies of the CNS. Based on our results, dosing is optimized. Similar experiments are performed with Th1 and Th17 polarized cells from MOG₃₅₋₅₅ C57BL/6 mice to determine whether results are consistent across mouse strains and autoantigens.

It is contemplated that administration of MAdCAM-1-Fc or ΔMAdCAM-1-Fc, but not vMAdCAM-1-Fc, will prevent both Th17 and Th1 induced EAE, though suppression of Th1 mediated disease might require higher doses of the reagent. If MAdCAM-1-Fc is impotent against Th driven EAE, it is determined whether it is responsive to blockade of other adhesion molecules (such as P-selectin ligand).

### Example 4

### Reactivation of Latent Viral Infection in Mice Treated with MAdCAM-1-Fc Versus VCAM-1-Fc

PML is a rare but potentially catastrophic side effect of natalizumab therapy. Up to 90% of adults are seropositive for the JC polyoma virus, the infectious cause of PML. Acute infection is usually asymptomatic. Afterwards the virus becomes latent in the spleen, the reticuloendothelial system and the medulla of the kidney. Most studies indicate that latent JC virus is undetectable in brain tissue. Years after the initial infection, reactivation can occur in the kidneys and bone marrow, usually in the setting of immunosuppression. It is not known how the reactivated virus is transmitted to the brain. It is believed that infected B cells (a major reservoir for dormant JC virus) cross the blood-brain-barrier and pass the infection to glial cells. Virus replication ensues and ultimately results in destructive infection of oligodendrocytes and consequent demyelination. The lesions may occur in any location in the white matter, and they range from 1 mm to several centimeters in size. The histopathologic hallmarks of PML include a triad of multifocal demyelination, hyperchromatic enlarged oligodendroglial nuclei, and enlarged bizarre astrocytes with lobulated and hyperchromatic nuclei. Electron microscopy reveals JC virus in the oligodendroglial cells. Progressive focal neurologic deficit is the clinical hallmark. Weakness and disturbance of speech are most common symptoms. Other symptoms include cognitive abnormalities, headaches, gait disorders, visual impairment, and sensory loss.

PML is most frequently seen in AIDS. Approximately 2-5% of AIDS patients develop the infection. There is no known cure. The median survival of patients with PML as a complication of AIDS is 6 months. In 10% of patients, survival exceeds 12 months. Among the 8 MS patients who developed PML, one died. In the other 7 cases the diagnosis was made, and natalizumab therapy was withdrawn, expeditiously. All 7 survived but the majority suffered permanent new disability by report. Current hypotheses regarding PML pathogenesis posit that virus recrudescence occurs when normal immune surveillance of the CNS declines via a failure of virus-reactive lymphocytes to gain access to the brain and spinal cord.

It is contemplated that administration of MAdCAM-1-Fc will not suppress protective immunity to the same extent as VCAM-Fc due to the relatively restricted range of expression of α4β7 on leukocytes and of MAdCAM-1 on endothelium. The alphavirus, Sindbis virus (SV), causes acute encephalitis in mice followed by clearance of infectious virus from CNS tissue over an 8-10 day period, even though viral RNA can be recovered by PCR-based methods for many months thereafter. Viral clearance is mediated by an antigen-specific immune response, and low numbers of antiviral immune effectors persist within the CNS for months after the infectious virus is cleared. This persistent cellular infiltrate is believed to prevent viral reactivation; systemic immune suppression with cyclophosphamide allows for recrudescence of infectious virus from the CNS months later. Thus, SV infection of mice provides a model in which targeted anti-adhesion molecule therapies can be assessed for their potential to suppress host responses against acute CNS viral infection and to promote reactivation of latent virus and CNS infection.

Mice are infected with 10³ plaque-forming units of SV via a direct intracerebral route and then treated daily with either optimized doses of MADCAM-Fc, vMAdCAM-1-Fc, VCAM-Fc or anti-α4 integrin monoclonal antibodies. Brain and spinal cord tissue are obtained from triplicate mice on days 1, 3, 5, 7, and 9 post-infection for virus titration assays, and on days 1, 3, 5, 7, 9, 14, 17, 21, and 28 to measure viral RNA levels by qPCR. The effect of such interventions on the entry of total and virus-specific B cells into the CNS is investigated using flow cytometry and a previously established ELISPOT-based assay to enumerate virus-specific B cells, respectively. In this way any detrimental effects of MADCAM-Fc on the adaptive immune response to acute viral infection of the CNS is determined.

Despite prompt and effective clearance of infectious virus from the CNS of mice with acute SV encephalitis via an antigen-specific humoral immune response, viral RNA can still be detected in the brain many months later. Reactivated infection can occur with systemic immune suppression, and the continued presence of virus-specific B cells in the brain after recovery likely prevents this reactivation from occurring. The present invention is not limited to a particular mechanism. Indeed, an understanding of the mechanism is not necessary to practice the present invention. Nonetheless, it is postulated that these B cells must continually traffic from the periphery into the CNS to control infection and, consequently, anti-adhesion molecule therapies pose a risk of late SV reactivation. To investigate such a possibility, mice that have recovered from acute encephalitis are treated with either optimized doses of MADCAM-Fc, vMAdCAM-Fc, VCAM-Fc or anti-α4 integrin monoclonal antibodies. At defined intervals thereafter, brains and spinal cords are collected from triplicate mice at each time point to measure viral RNA levels by qPCR, and if increases are detected, then infectious virus by plaque titration assays. The effects such interventions on the number of total and virus-specific B cells in the CNS is also investigated using flow cytometry and ELISPOT-based assay, respectively.

It is contemplated that systemic MADCAM-Fc or anti-adhesion molecule antibody treatment of mice with acute SV encephalitis will reduce the infiltration of lymphocytes into the CNS thereby delaying (but not preventing) clearance of infectious virus from the CNS.

It is further contemplated that systemic VCAM-Fc treatment of mice with latent SV infection of the CNS will trigger a measurable rise in the amount of viral RNA detected in the brain compared to control treated animals resulting in neuronal and glial damage. By contrast, mice treated with MAdCAM-1 Fc will continue to control latent SV infection.

### Example 5

This Example describes the efficacy of human MadCAM in the EAE animal model. FIG. 11 shows that human MAdCAM-Fc accelerates recovery from acute EAE when administered following disease onset. SJL mice were immunized with PLP₁₃₉₋₁₅₁ in CFA. Mice were divided into 2 groups matched for clinical score on day 11 post-immunization (n=5/group). Each group was treated with either human MAdCAM-Fc or control Fc protein on days 13, 15 and 17 post-immunization (1 mg/mouse i.v.). Animals were rated for degree of neurological disability on a daily basis by an examiner blinded to the treatment of each group. (*p<0.05).

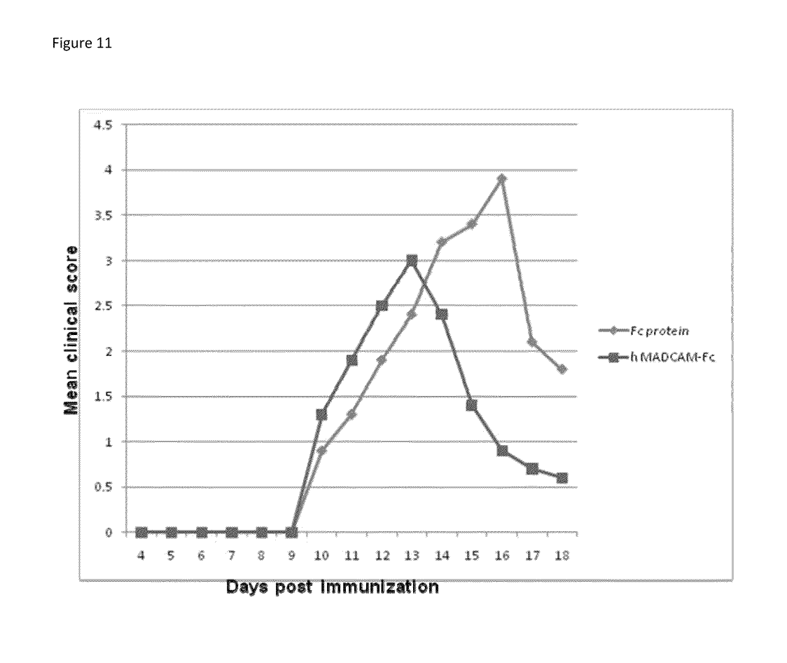

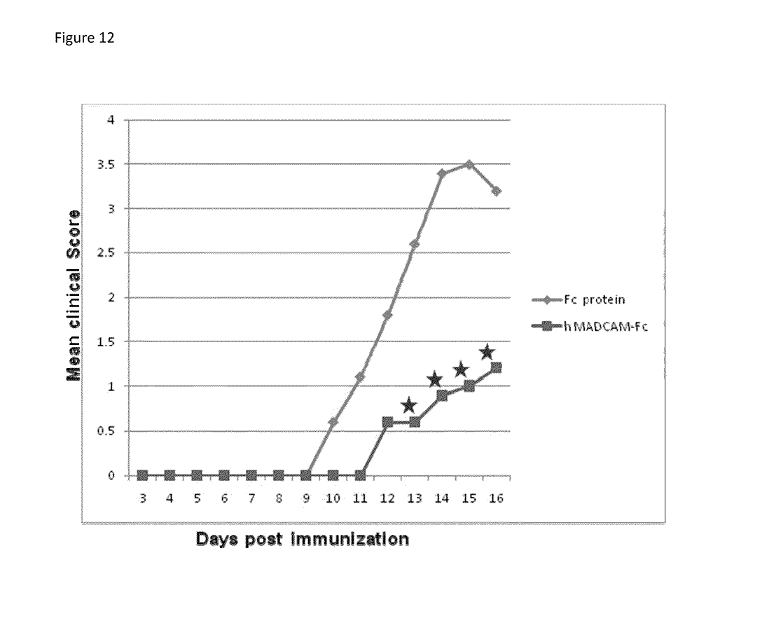

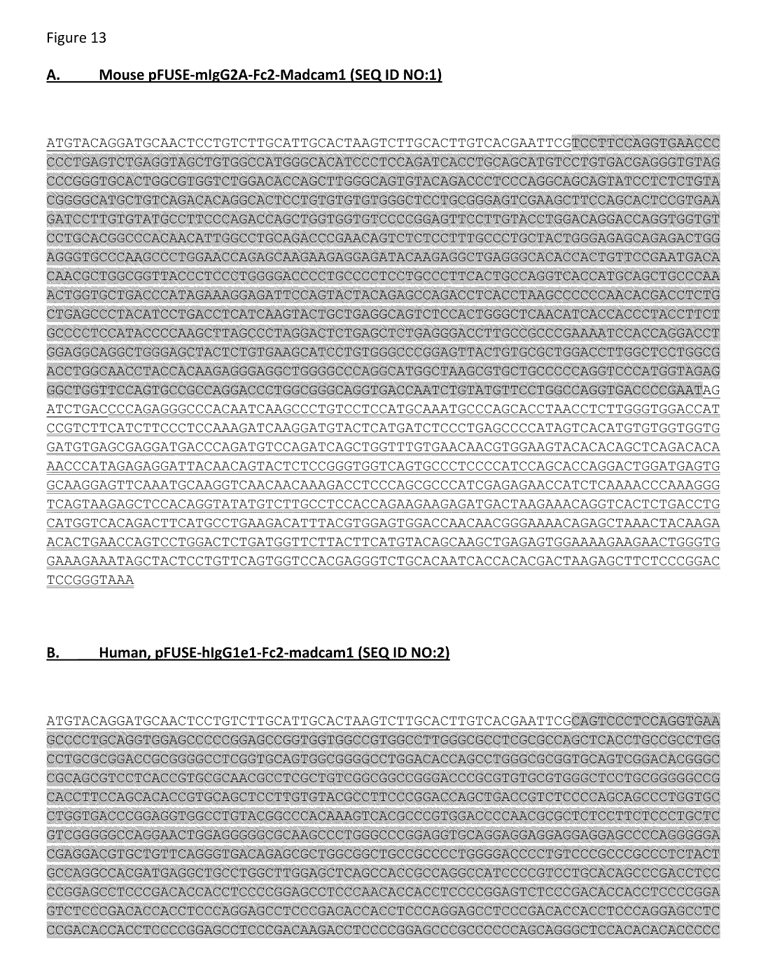

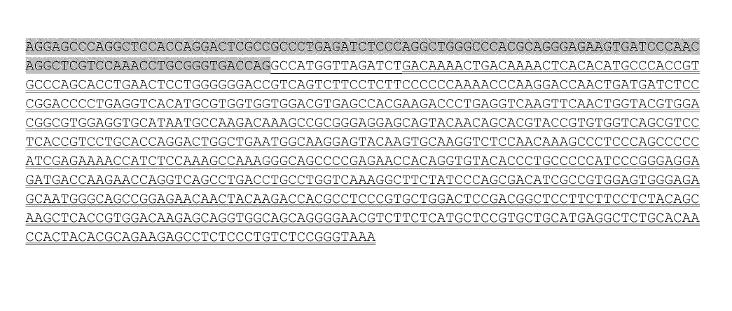

FIG. 12 shows that human MAdCAM-Fc suppresses EAE induced by active immunization. SJL mice were immunized with PLP₁₃₉₋₁₅₁ in CFA. Mice were divided into 2 groups (n=5/group) for treatment with either human MAdCAM-Fc or control Fc protein on days 7, 9, 11, and 13 post-immunization (1 mg/mouse i.v.). Animals were rated for degree of neurological disability on a daily basis by an examiner blinded to the treatment of each group. (*p<0.05).

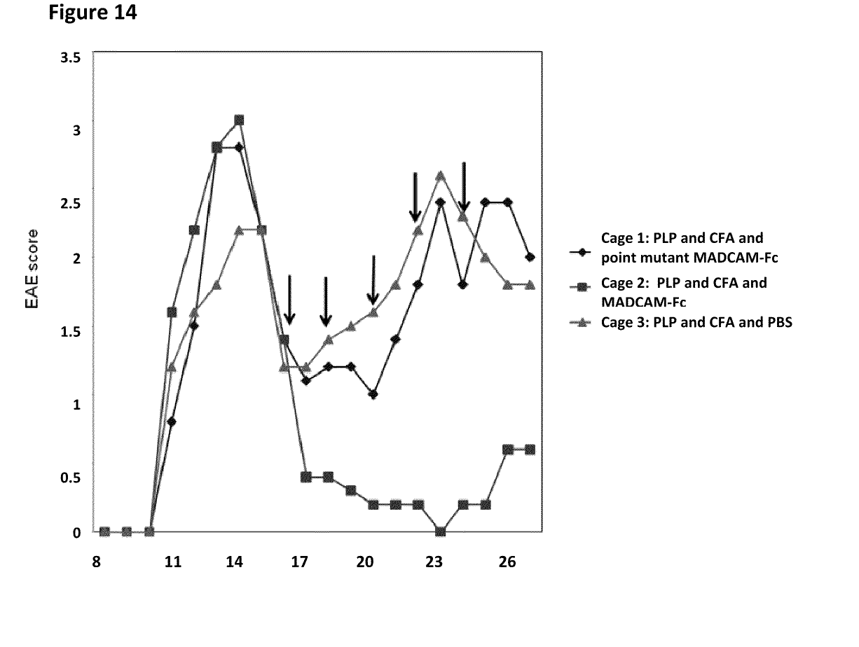

In further experiments, wild type MADCAM-Fc was mutated in its ligand binding domain and the mutant's effect on EAE in the animal model was measured. Results are shown in FIG. 14 and demonstrate that the mutant protein was not able to prevent EAE.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be within the scope of the following claims.

1. A pharmaceutical composition comprising an FC-Fusion protein that inhibits at least one activity of a mucosal addressin cell adhesion molecule-1 (MAdCAM) protein.

2. The composition of claim 1, wherein said FC-fusion protein has the amino acid sequence encoded by SEQ ID NO: 2.

3. The composition of claim 1, wherein said composition reduces or eliminates symptoms of a neuroinflammatory condition.

4. The composition of claim 1, wherein said composition prevents relapses of a neuroinflammatory condition.

5. The composition of claim 1, wherein said neuroinflammatory condition is multiple sclerosis.

6. A method of treating a neuroinflammatory condition, comprising:
Administering a composition that inhibits at least one activity of a MAdCAM or β7 integrin protein to a subject diagnosed with a neuroinflammatory condition under conditions such that symptoms of said neuroinflammatory condition are reduced or eliminated.

7. The method of claim 6, wherein said neuroinflammatory condition is multiple sclerosis.

8. The method of claim 6, wherein said composition is selected from the group consisting of a MAdCAM FC-Fusion, an anti-MAdCAM antibody, a small molecule, an siRNA that inhibits the expression of MAdCAM and an antisense nucleic acid that inhibits the expression of MAdCAM.

9. The method of claim 6, wherein said composition is selected from the group consisting of an anti-β7 integrin antibody, a small molecule, an siRNA that inhibits the expression of β7 integrin and an antisense nucleic acid that inhibits the expression of β7 integrin.

10. The method of claim 8, wherein said composition is a MAdCAM FC-Fusion.

11. The method of claim 10, wherein said FC-fusion protein has the amino acid sequence encoded by SEQ ID NO: 2.

12. The method of claim 6, wherein said composition accelerates recovery of said subject from symptoms of said neuroinflammatory condition.

12. The method of claim 6, wherein said composition prevents relapses of symptoms of said neuroinflammatory condition.
